(19) United States
(12) Patent Application Publication
(10) Pub. No.: US 2002/0016443 A1
(43) Pub. Date: Feb. 7, 2002

(54) Antiproliferative factor

(76) Inventors: Susan K. Keay, Ellicott City, MD (US); John W. Warren, Baltimore, MD (US); Michael Kleinberg, Baltimore, MD (US); Michael K. Hise, Columbia, MD (US)

Correspondence Address: David L. Marks, University of Maryland, Baltimore Office of Research & Dev., 515 West Lombard Street, Suite 500, Baltimore, MD, 21201-1602, US

(21) Appl. No.: 09/839,859

(22) Filed: Apr. 21, 2001

Related U.S. Application Data
(63) Continuation-in-part of application No. 09/307,686, filed on May 10, 1999, now abandoned.
Application No. 09/307,686 is a division of application No. 08/944,202, filed on Oct. 3, 1997, now Pat. No. 5,962,645.
(60) Provisional application No. 60/027,646, filed on Oct. 4, 1996.
(60) Provisional application No. 60/218,272, filed on Jul. 13, 2000.
(60) Provisional application No. 60/232,911, filed on Sep. 15, 2000.

(51) Int. Cl. G01N 33/53; G01N 33/567; A61K 39/395; C12N 15/12; C12P 21/00

(52) U.S. Cl. 530/350; 424/130.1; 435/7.2; 514/2; 536/23.5

(57) ABSTRACT

The invention relates to a novel antiproliferative factor (APF) present in urine of patients with interstitial cystitis (IC). APF is useful as a marker for disease activity and its antagonists are useful as therapeutic medicaments for IC and other conditions associated with elevated APF. APF and its agonists are useful in the treatment of diseases associated with cell proliferation, such as bladder cancer.

This application is a CIP of U.S. patent application Ser. No. 09/307,686 filed on May 10, 1999, now abandoned. U.S. Pat. No. 09/307,686 is a Divisional of U.S. Pat. No. 5,962,645 issued on Oct. 5, 1999 and filed on Oct. 3, 1997 as U.S. patent application Ser. No. 08/944,202 which claims priority to U.S. patent application Ser. No. 60/027,646 filed on Oct. 4, 1996 (now abandoned). This application claims priority to U.S. patent application Ser. No. 09/307,686, U.S. Pat. No. 5,962,645, and U.S. patent application Ser. No. 60/027,646. This application also claims priority to and is related to U.S. patent application 60/218,272 filed on Jul. 13, 2000 and to U.S. patent application Ser. No. 60/232,911 filed on Sep. 15, 2000.

The development of the present invention was supported by the University of Maryland, Baltimore, Md. and by funding from the National Institutes of Health (NIH) under grant number DK 44818 and DK 970050. The United States Government may have rights in certain aspects of the invention herein to the extent provided by the terms of the NIH grant. The research for this invention was also support by a grant from the Interstitial Cystitis Association (ICA-990558).

1. FIELD OF THE INVENTION

The invention relates to a novel antiproliferation factor found in subjects with interstitial cystitis; to methods for isolating the antiproliferation factor; to methods for using the antiproliferation factor in the treatment of conditions characterized by cellular proliferation; and to methods for diagnosing and treating interstitial cystitis.

2. BACKGROUND OF THE INVENTION

Art relating to the background of the invention is reviewed in the ensuing sections.

2.1 Interstitial Cystitis

Interstitial cystitis (IC) is a chronic bladder disorder which affects up to 450,000 women in the United States; approximately one-tenth as many men also suffer from this condition.¹ IC typically presents with a rapid onset, with pain, urgency and frequency of urination and cystoscopic abnormalities including petechial hemorrhages (glomerulations) or ulcers that extend into the lamina propria (Hunner's ulcers).² Certain features of the bladder epithelium suggest that the epithelial barrier is abnormal in IC. For example, the bladder mucin layer is sometimes damaged,³ the bladder epithelium can be denuded resulting in ulceration,⁴ and intraurothelial Tamm-Horsfall protein is sometimes present.⁵ The rapid onset of IC is followed by a chronic course with partial remissions and re-exacerbations, which can continue for up to 30 years.⁶ No etiology for IC has yet been identified, and no empiric treatment has proven to be reliably efficacious. Accordingly, there is a need for compositions and methods useful in the treatment of IC.

Additionally, the diagnosis of IC currently requires cystoscopy and bladder biopsy, with either of two distinct mucosal abnormalities (Hunner's ulcers or glomerulations) being diagnostic of this disorder. Consequently, there is a need for a faster, less invasive method for diagnosing IC.

2.2 Bladder Cancer and Other Proliferative Diseases

A tumor (i.e., a neoplasm) is a mass resulting from abnormal, uncontrolled cell growth. Tumors can be benign or malignant. Benign tumors generally remain localized. The term “malignant” generally means that the tumor can invade and destroy neighboring body structures and spread to distant sites to cause death.⁷

Treatment options for cancer include, surgery, chemotherapy and radiation treatment. Such options are commonly ineffective or present serious side effects. Accordingly, there is a need for new drugs for use in the treatment of cancer.

Bladder cancer is one of the most common malignancies. Approximately 50,000 new cases diagnosed each year in the United States, and more than 10,000 cancer deaths are attributed to bladder cancer. In North America, transitional cell carcinomas account for more than 90% of bladder malignancies. About 6% are squamous cell tumors, and 1% are adenocarcinomas.⁸

The majority of patients (approximately 75%) with transitional cell bladder cancer present with superficial papillary cancer. About 10% progress to invasive (involving the muscularis propria; stage T2 and higher) cancer.⁹

The 5-year survival of patients presenting with stage T2 or higher bladder cancer is only about 50% to 60%. The vast majority of patients with invasive bladder cancer who die of the disease do so from systemic metastases. The implementation of systemic chemotherapy has been an area of extensive clinical research because of the significant failure rate, radical cystectomy notwithstanding¹⁰. There is a significant need in the art for new more effective therapeutic agents for treating bladder cancer.

### 3. SUMMARY OF THE INVENTION

The invention provides a purified anti-proliferative factor (APF) isolated from a composition comprising materials secreted from bladder epithelial cells from a subject having interstitial cystitis and functional equivalents of APF. The invention also provides a pharmaceutical composition comprising APF or a functional equivalent thereof and a pharmaceutically acceptable carrier.

The invention provides a purified anti-proliferative factor (APF) isolated from a composition comprising materials present in the urine from a subject having interstitial cystitis and functional equivalents of APF. The invention also provides a pharmaceutical composition comprising APF or a functional equivalent thereof and a pharmaceutically acceptable carrier.

APF exhibits absorbance at approximately 215 and 280 nm. The molecular mass of APF in one embodiment is from about 1 to about 10 kDa, preferably 1.5 to 2.7 kDa, and more preferably either 1.7 to 1.8 kDa or 2.4 to 2.5 kDa, depending on the method by which the mass is measured. It will be appreciated by those of skill in the art that the apparent molecular mass may vary depending on the attachment of various substituents. Other identifying characteristics are more fully discussed in the Detailed Description of the Invention (Section 6) and in the subsequent examples (Section 7).

The invention includes methods for isolating APF. For example, APF may be obtained from urine of subjects with interstitial cystitis. APF may also be isolated from culture medium from a culture of epithelial bladder cells from a subject with interstitial cystitis.

Another aspect of the invention relates to a purified APF isolated from a composition comprising materials produced by bladder epithelial cells from a subject having interstitial cystitis. Purified APF can also be isolated from a composition comprising materials produced by bladder epithelial cells from a subject exhibiting decreased levels of heparin-binding epidermal growth factor-like growth factor, as compared to levels of heparin-binding epidermal growth factor-like growth factor in a sample of subjects with bacterial cystitis or in a sample of subjects without interstitial cystitis nor bacterial cystitis.

The invention also relates to a bladder epithelial cell culture that produces APF. The cell culture is preferably originated from a bladder biopsy of a subject with interstitial cystitis. The cells are preferably immortalized.

In another aspect, the invention provides an active fraction of urine from a subject with interstitial cystitis, the fraction exhibiting antiproliferative activity.

The invention also relates to a method for inhibiting epithelial cell HB-EGF production. The method comprises contacting epithelial cells with HPLC-purified APF from a composition comprising substances secreted from bladder cells or from a composition comprising substances present in the urine of subjects with interstitial cystitis.

In another embodiment, the invention relates to a method for identifying the structure of an APF from urine of subjects with interstitial cystitis, the method comprising analyzing the structure of APF using one-dimensional and/or two-dimensional NMR.

In another aspect, the invention relates to a method for downregulating HB-EGF production by a cell comprising bringing the cell into contact with an APF isolated from a composition comprising materials secreted from bladder cells of a subject having interstitial cystitis or from a composition comprising materials present in the urine of subjects with interstitial cystitis.

The invention also relates to a method for inhibiting proliferation of cells comprising contacting the cells with an APF isolated from a composition comprising materials secreted from bladder cells of a subject having interstitial cystitis or from a composition comprising materials present in the urine of subjects with interstitial cystitis.

The invention also relates to a method for regulating production of HB-EGF, EGF, IGF1 and IGFBP3 in a subject comprising administering to the subject an APF isolated from a composition comprising materials secreted from bladder cells of a subject having interstitial cystitis or from a composition comprising materials present in the urine of subjects with interstitial cystitis.

The present invention also relates to a method for diagnosing interstitial cystitis in a patient which comprises assaying for the presence of APF.

In another aspect, the invention relates to therapeutic medicament comprising APF or an agonist of APF for treating a disease associated with an increase in cell proliferation such as tumorigenesis or cancer.

4. DEFINITIONS

The term “APF compounds” is used herein to refer to APF and its functional equivalents. APF compounds can be obtained from humans, animals, or cells isolated from humans or animals. APF compounds can also be made using apparatus appropriate for the synthetic production of APF.

The term “anti-APF compounds” is used herein to refer to compounds which bind to APF or otherwise inhibit the antiproliferative effect of APF.

The term “therapeutic compound” is used herein to refer to both APF compounds and anti-APF compounds.

The term “functional equivalent” as used herein refers to a polypeptide sequence comprising APF, or comprising a fragment, analog, derivative or truncation isoform of APF. Functional equivalents also include salts or complexes, for example, APF, an APF fragment, an APF analog, an APF derivative or an APF truncation isoform, in a salt or complex. Functional equivalents retain some or all of the biological activity of the corresponding native APF.

A “therapeutically effective” amount or dose is an amount or dose which prevents or delays the onset or progression of an indicated disease or other adverse medical condition. The term also includes an amount sufficient to arrest or reduce the severity of an ongoing disease or other adverse medical condition, and also includes an amount necessary to enhance normal physiological functioning.

As used herein, “treatment” of a disease or other adverse medical condition, should be broadly interpreted based on the therapeutic effects described herein as variously including active, causal, conservative, medical, palliative, prophylactic, and/or symptomatic treatment, treatment which delays the onset or progression of the disease or other adverse medical condition, as well as treatment which arrests or reduces the severity of an ongoing disease or other adverse medical condition.

As used herein, a “pharmaceutically acceptable” component (such as a salt, carrier, excipient or diluent) of a formulation according to the present invention is a component which (1) is compatible with the other ingredients of the formulation in that it can be combined with the therapeutic compound (e.g., an APF compound) without eliminating the biological activity thereof, and (2) is suitable for use in non-human animals or humans without undue adverse side effects (e.g., toxicity, irritation, and allergic response). Side effects are “undue” when their risk outweighs the benefit provided by the pharmaceutical composition.

As used herein, a “pharmaceutically acceptable” with reference to the degree of purity of an APF compound or nucleic acid indicates that an APF compound or nucleic acid (1) is free of contaminating materials that would eliminate the biological activity of an APF compound or nucleic acid; and (2) is free of contaminating materials that would render an APF compound or nucleic acid unsuitable for administration to non-human animals or humans by causing undue adverse side effects (e.g., toxicity, irritation, and allergic response). Side effects are “undue” when their risk outweighs the benefit provided by the APF compound or nucleic acid.

The term “substantially pure” when used in reference to an APF compound is defined herein to mean an APF compound or nucleic acid that is substantially free from other contaminating proteins, nucleic acids, and other biologicals derived from an original source organism, recombinant DNA expression system, or from a synthetic procedure employed in the synthesis or purification of an APF compound of nucleic acid (e.g., chromatography reagents and polymers, such as acrylamide or agarose). Purity may be assayed by standard methods. Purity evaluation may be made on a mass or molar basis.

### 5. BRIEF DESCRIPTION OF THE DRAWINGS

FIG. 1A: Inhibition of cell proliferation by IC patient urine specimens, asymptomatic controls, and bacterial cystitis patients (³H-thymidine incorporation).

FIG. 1B: Inhibition of BrdU incorporation in normal adult human bladder epithelial cells by IC patient urine specimens and asymptomatic controls.

FIG. 1C: Inhibition of cell proliferation by IC patient urine specimens, asymptomatic controls, patients with bacterial cystitis (BC), or patients with vulvovaginitis (VV) (³H-thymidine incorporation).

FIG. 1D: Net inhibition of HBE cell proliferation by IC patient #1 and control urine specimens.

FIG. 1E: Net inhibition of HBE cell proliferation by IC patient #2 and control urine specimens.

FIG. 2: Inhibition of T24 carcinoma cell proliferation by IC patient urine specimens.

FIG. 3: Inhibition of human bladder cell proliferation by low molecular weight fractions from IC urine.

FIG. 4: Separation of urinary components by HPLC.

FIG. 5: Separation of urinary components by ion exchange chromatography.

FIG. 6: Separation of low molecular weight urinary components by hydrophobic interaction chromatography.

FIG. 7A: Separation of low molecular weight urinary components by HPLC.

FIG. 7B: Determination of APF purity by HPLC.

FIG. 8: MALDI-TOF mass spectrometric analysis of purified APF.

### 6. DETAILED DESCRIPTION OF THE INVENTION

For ease of reference, and not by way of limitation, the Detailed Description of the Invention is divided into the ensuing sections.

6.1 Identifying Characteristics of APF

The inventors have discovered that urine from IC patients inhibits the proliferation of normal bladder epithelial cells in vitro, and that this antiproliferative activity is due to a low molecular weight, heat stable peptide that can be used as an indicator for this disease. This novel factor is referred to herein as “APF.” APF is found in, and can be isolated from, natural sources, such as urine of IC patients, referred to herein as “IC urine.” APF is useful for inhibiting proliferation of cells, such as primary normal adult human bladder epithelial cells or cells derived from bladder tumors, and in the diagnosis of interstitial cystitis.

APF is identified by a number of identifying characteristics, discussed below. Any one or more of these identifying characteristics may be used to identify APF or to identify fractions of a source of APF (e.g., fractions of IC urine). Preferably multiple characteristics are used.

6.1.1 Structural Characteristics of APF

APF is generally characterized by the following structural characteristics:

blocked N-terminal amino acid;

presence of a gly-gly-ala modified segment;

presence of an aromatic group, possibly tyrosine;

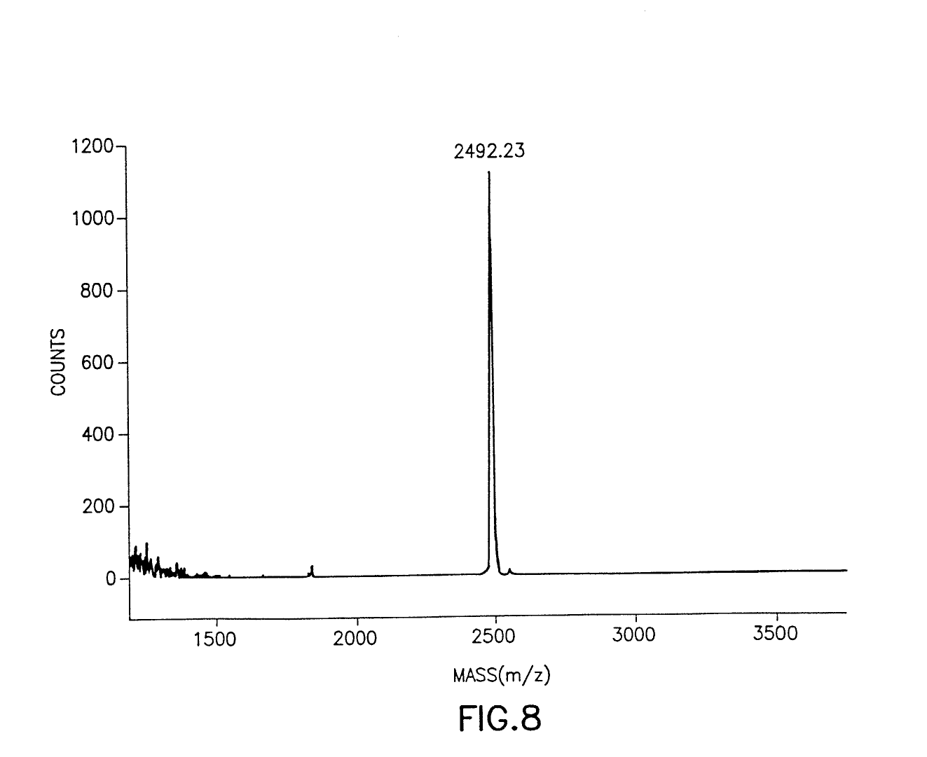

a mass spectrometry profile corresponding to the profile set forth in FIG. 8;

the following amino acid composition: 2 asparagines/aspartic acids, 1 threonine, 2 serines, 4 glutamines/glutamic acid, 1 proline, 4 glycines, 2 alanines, 1 valine, 1 isoleucine, 2 leucines, 1 tyrosine, 1 phenylalanine, 1 lysine, 1 arginine, and at least one cysteine;

a cysteine coupled to a leucine;

a tyrosine coupled to a serine;

a possible disulfide bond between two cysteines;

6.1.2 Functional Characteristics of APF and APF Fractions

APF and active fractions of a source of APF (“APF fractions”) are also characterized by functional attributes. The functional attributes of APF and APF fractions of a source of APF can be demonstrated using an assay for cellular proliferation. For example, antiproliferative activity can be demonstrated by inhibition of ³H-thymidine or BrdU incorporation in a cell culture. APF and APF fractions effect significant inhibition in such assays as compared to controls. APF purified to homogeneity or APF fractions can therefore be identified functionally using such cellular proliferation assays.

APF is also characterized by an ability to inhibit T24 bladder carcinoma cell proliferation. Therefore, APF and APF fractions can be identified functionally using assays for such antiproliferative activity with respect to T24 bladder carcinoma cells.

6.1.3 Physical/Chemical Characteristics of APF and APF Fractions

Additionally, APF is characterized by a molecular mass from about 1 to about 10 kDa, preferably 1.5 to 2.7 kDa, and more preferably either 1.7 to 1.8 kDa or 2.4 to 2.5 kDa, depending on the type of equipment used to perform the analysis. It will be appreciated by those of skill in the art that the molecular mass may vary, depending on the presence or absence of various carbohydrate or lipid moieties which may be attached to the amino acids of APF. Nevertheless, molecular mass remains an important identifying characteristic, which can be used to narrow fractions of urine in which APF resides.

APF is also characterized by stability in a freeze-thaw cycle, with approximately 18.5+/−8.2% loss of activity. Consequently, fractions of IC urine in which APF resides can be identified by subjecting the fractions to a freeze-thaw cycle to determine the loss of activity following a freeze-thaw cycle, the fraction containing the APF having a loss of activity of between 10.3% and 26.7%.

APF is further characterized by exhibiting low stability in the presence of trypsin. Loss of activity when incubated with trypsin is typically about 87.5+/−26.7% as compared to control APF exposed to corresponding incubation conditions in the absence of trypsin. Thus, incubation with trypsin and subsequent analysis for loss of activity can be used to identify fractions of IC urine in which APF is present.

6.2 Purifying APF

APF can be isolated from a composition comprising materials in the urine of a subject having interstitial cystitis or from a composition comprising materials secreted from bladder epithelial cells from a subject having interstitial cystitis. IC patients may be identified using the 1989 National Institute of Diabetes and Digestive and Kidney Diseases diagnostic criteria for interstitial cystitis. IC patients may also be identified by decreased levels of heparin-binding epidermal growth factor-like growth factor, as compared to levels of heparin-binding epidermal growth factor-like growth factor in a sample from normal population.

APF may also be isolated from culture medium from a culture of bladder epithelial cells that were isolated from a subject having interstitial cystitis. It is preferable that the bladder epithelial cells be immortalized.

In general, purification of APF can be performed by preparing <10 kDa fractions from a source of APF (e.g., from urine specimens of IC patients or culture medium of a culture of bladder epithelial cells isolated from IC patients) using filters for fractionation of specimen by size such as “CENTRIPREP” filters (Amicon, Beverly, Mass.). APF can then be further purified from these fractions by known protein purification methods which separate proteins based on properties such as charge, hydrophobicity, and size. Examples of suitable techniques include ion-exchange and hydrophobic interaction chromatography and high performance liquid chromatography (HPLC).

Thus, for example, APF can be isolated by a method comprising, in order, the following steps: (a) obtaining a <10,000 fraction of urine from a subject with interstitial cystitis or culture medium of a culture of bladder epithelial cells isolated from IC patients; (b) obtaining, by ion-exchange chromatography, a functionally active sub-fraction from the fraction of (a); (c) obtaining, by hydrophobic interaction chromatography, a functionally active subfraction of the fraction of (b); and isolating, by HPLC, the functionally active APF from the subfraction of (c).

Another example, APF can be isolated by a method comprising: (a) loading onto a sepharose column a <10,000 fraction of urine from a subject with interstitial cystitis or culture medium of a culture of bladder epithelial cells isolated from IC patients; (b) eluting components of the fraction; (c) testing each component for the ability to inhibit proliferation of normal bladder epithelial cells, immortalized bladder epithelial cells or bladder cancer cells. The ability to inhibit proliferation of normal bladder epithelial cells, immortalized bladder epithelial cells or bladder cancer cells can be determined by ³H-thymidine incorporation (by way of example).

At each step in the isolation process, ³H-thymidine or BrdU incorporation can be employed as assays to detect fractions of which comprise APF. APF preferably produces a mean % change in ³H-thymidine incorporation in bladder cells of greater than 2 standard deviations from the mean incorporation of control cells incubated with cell medium alone.

The active fractions of urine from a subject with interstitial cystitis are also an aspect of the present invention. Active fractions may be obtained by a method comprising a wide variety of separation techniques known in the art. Examples of suitable procedures include ion-exchange chromatography, hydrophobic interaction chromatography, and HPLC.

APF may be isolated from a composition comprising materials produced by bladder epithelial cells from a subject exhibiting decreased levels of heparin-binding epidermal growth factor-like growth factor, as compared to levels of heparin-binding epidermal growth factor-like growth factor in a sample of asymptomatic controls or subjects with bacterial cystitis.

APF may be isolated from urine of subjects that exhibit interstitial cystitis or decreased levels of heparin-binding epidermal growth factor-like growth factor, as compared to levels of heparin-binding epidermal growth factor-like growth factor in a sample of asymptomatic controls or subjects with bacterial cystitis.

APF may be isolated from a composition comprising materials produced by bladder epithelial cells from a subject exhibiting increased levels of one or more factors selected from the group consisting of epidermal growth factor, insulin-like growth factor 1, and insulin-like growth factor binding protein 3, as compared to asymptomatic controls or subjects with bacterial cystitis.

The invention further provides for a bladder epithelial cell culture which produces APF. The cell culture is preferably originated from a bladder biopsy of a subject with interstitial cystitis. The cells are preferably immortalized. The culturing may be accomplished using a standard cross-flow filtration system in which the culture medium containing the cultured cells is circulated past a filter membrane which permits APF to traverse the filter membrane while retaining the cells. Moreover, a resin comprising antibodies with specificity for APF may be provided across the filter membrane from the culture fluid and cells to increase APF concentration gradient across the membrane. Other cross-flow filtering systems may be provided to separate APF from contaminating substances which cross the filter membrane. Where such contaminants are nutrients, they may be flowed back into the culture medium.

Briefly, the inventors purified APF as follows: A <10,000 fraction of urine from a subject with interstitial cystitis or culture medium of a culture of bladder epithelial cells isolated from IC patients is obtained. A a functionally active sub-fraction from the first fraction is obtained by ion-exchange chromatography. Then a functionally active subfraction of the fraction of first sub-fraction is obtained by hydrophobic interaction chromatography. Next functionally active APF from the second sub-fraction by HPLC. Each purified fraction or subfraction was desalted by dialysis and the ability of a fraction to inhibit ³H-thymidine incorporation in human bladder epithelial (HBE) cells assessed as described below by performing the HBE cell proliferation assay; results obtained from IC patient urine fractions were compared to results obtained using urine from age-, race-, and sex-matched controls.

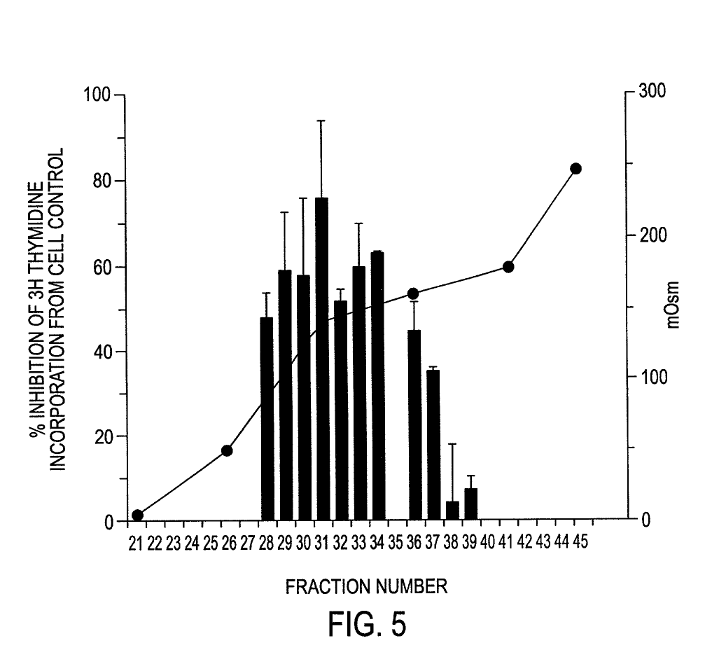

Anion exchange chromatography using a “MONO Q” sepharose column (functional group CH₂N⁺(CH₃)₃, Sigma, St. Louis, Mo.) was useful for partial preparative APF purification. The “MONO Q” matrix was suspended in 500 mM phosphate buffer (pH 7.0) and washed with 20 mM phosphate buffer (pH 7.0). The <10 kDa fractions of urine from large IC or control urine collections (500 ml each) were diluted 1:1 in 20 mM phosphate buffer and loaded onto the column at 4° C. overnight. Following a column wash with 20 mM phosphate buffer, protein was eluted with 1 M NaCl in 20 mM phosphate buffer (pH 7.0); fractions were diluted 1:40 in serum-free culture medium and applied to normal human bladder cells for the ³H-thymidine incorporation assay. By this method a wide peak of protein with antiproliferative activity was able to be eluted from the IC specimen which was not present in the control specimen (FIG. 5).

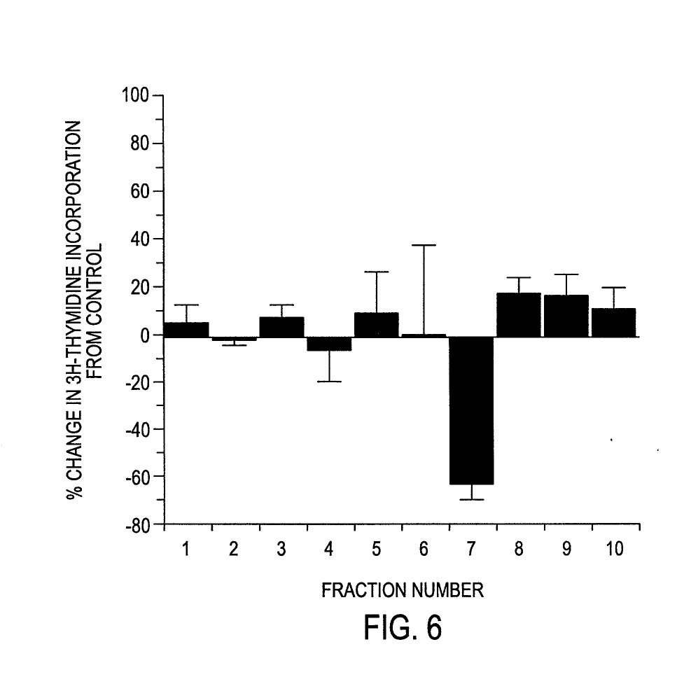

Hydrophobic interaction chromatography using a variety of matrices revealed the ability of a phenyl sepharose 6 fast flow (high sub) matrix (Pharmacia Biotech, Uppsala, Sweden) to be useful for further APF purification. The <10 kDa fraction from 10 ml of IC or control urine was adjusted to pH 6.0 with 10 N NaOH then diluted to 300 mOsm with double distilled H₂O. These preparations were then applied to phenyl sepharose 6 fast flow columns [which were suspended in 1M ammonium sulfate (pH 7.0)] at 4° C. ]. Protein was then eluted using 50 mM sodium phosphate buffer (pH 7.0). Run-through and eluted fractions were then dialyzed against phosphate buffered saline (pH 7.0) at 4° C. overnight, diluted 1:3 in serum-free culture medium, and applied to normal human bladder cells for the ³H thymidine incorporation assay. By this method a single fraction with antiproliferative activity was able to be obtained from IC urine that was not present in control urine (FIG. 6).

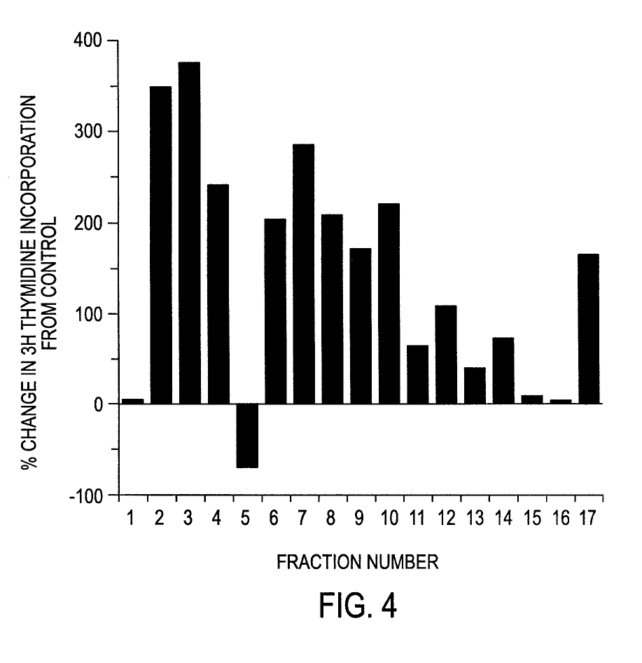

Reversed-phase high performance liquid chromatography (HPLC) was useful for further purification of the antiproliferative peptide (FIG. 4). The <10 kDa urine fractions from 2 IC patients and 2 controls were dialyzed against 10 mM sodium phosphate buffer, after which the dialysates were lyophilized, dissolved in water (50 fold concentration), and passed through 0.2 mm filters to remove particles. A 50 ml sample of each specimen was injected onto a C18 column (octadecyl aliphatic groups bonded to silica, Vydak, Hesperia, Calif.) and eluted with a 0-20% acetonitrile gradient [using 0.1% trifluoroacetic acid (TFA) in water (buffer A) and acetonitrile in 0.08% TFA (buffer B)]. Samples were dialyzed against phosphate buffered saline to remove acetonitrile and TFA, diluted 1:2 in serum-free cell culture medium, and incubated with HBE cells (48 hours at 37° C.). Analysis of subsequent ³H-thymidine incorporation indicated the presence of a single fraction containing antiproliferative activity in each IC specimen (data shown for one patient, FIG. 4) which included 2 protein peaks by optical density tracing at 215 nm. No inhibitory fraction was identified in the <10 kDa fraction from the age-, race- and sex-matched controls.

As a preliminary step for choosing an appropriate matrix and buffer system for ion exchange chromatography, the pI of the purified or partially purified APF was determined by isoelectric focusing, using a density gradient electrofocusing apparatus. The pI of APF was found to be in the range of 1.38-3.5. The pH curve was constructed from each sample, and the pH of each sample neutralized prior to performing the HBE cell proliferation assay; isoelectric focusing of the corresponding fraction from normal urine was also done and fractions were collected to serve as negative controls.

A sequential purification scheme which employs each of these three methods (preparative ion exchange chromatography followed by hydrophobic interaction chromatography followed by HPLC) was used to obtain highly purified APF from the low molecular weight (<10 kDa) fraction of urine from IC patients.

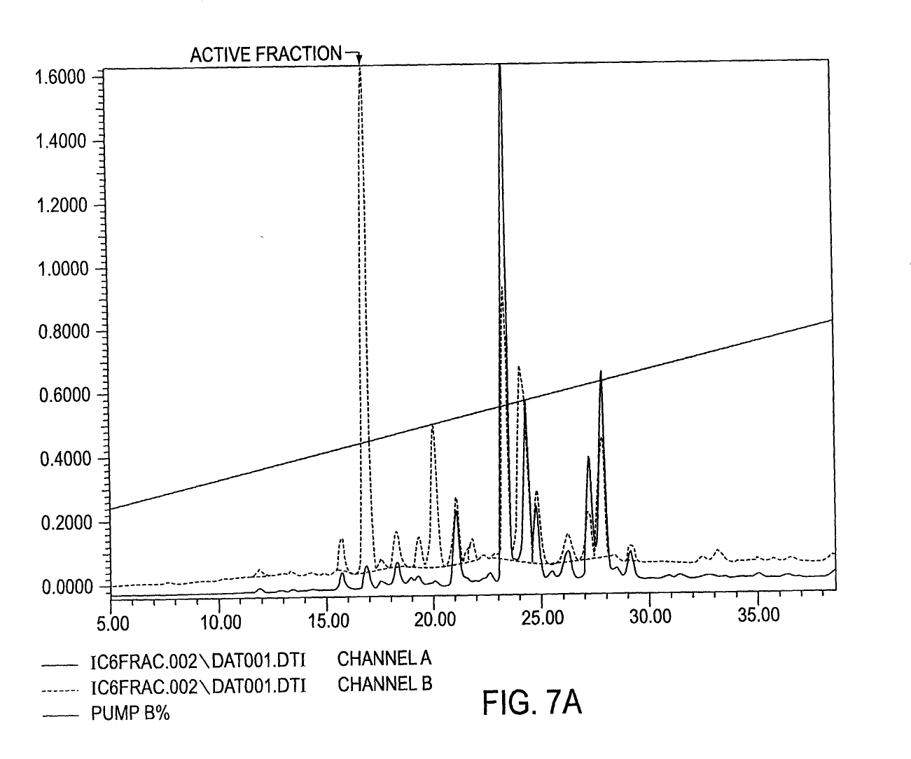

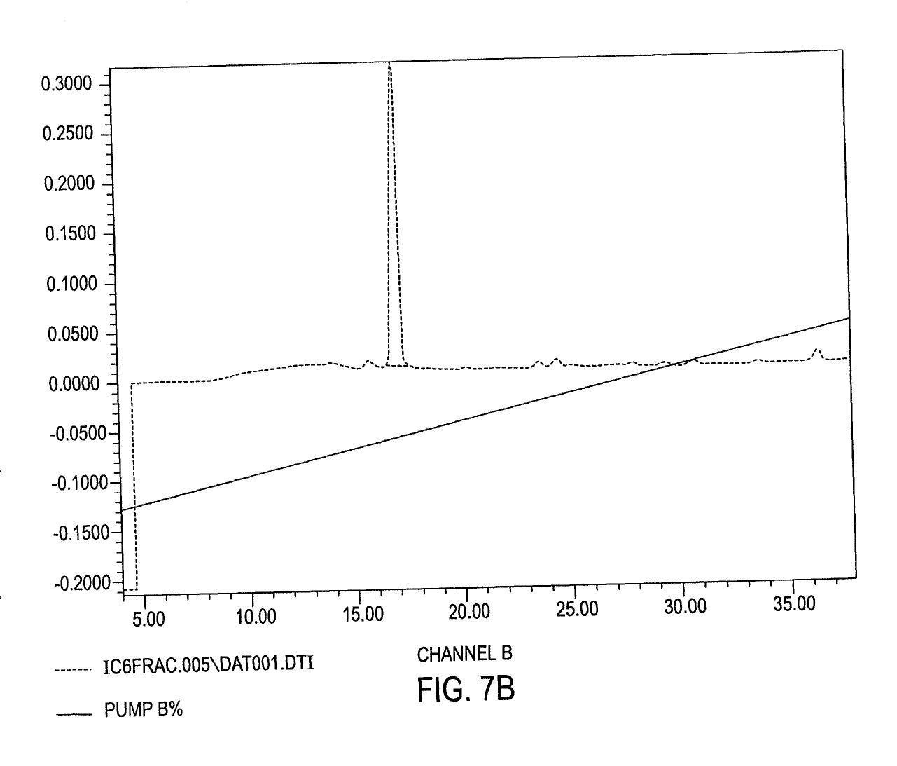

The purity of APF was confirmed by optical density tracing of protein in HPLC fractions (at 215 and 280 nm) (FIG. 7B). The molecular weight was determined by MALDI-TOF mass spectrometry and ion bombardment mass spectrometry to be between 1700 and 2500 daltons (8). MALDI-TOF mass spectrometric analysis was performed on a PerSeptive Biosystems (Framingham, Me.) Voyager. Mass calibration was done using as standards angiotensin I, ACTH (clip 1-17), ACTH (clip 18-39), ACTH (clip 7-38) and bovine insulin (PE Biosystems, Foster City, Calif.). A cyano-4-hydroxy-cinnaminic acid (Aldrich Chemical Co., Milwaukee, Wis.) at 10 mg/ml in 30% acetonitrile / 0.3% trifluoroacetic acid was used as the matrix. Using that methodology for MALDI-TOF mass spectrometry, the APF has a molecular mass of between 2.4 and 2.5 kDa.

Antiproliferative activity was easily measured from HPLC fractions of a small amount of urine (50 ml). HPLC experiments rendered a single peak which represented highly pure APF (FIG. 7B). HPLC/MS mass spectrometric analysis was also performed using a Hewlett-Packard 100 series HPLC, with 5% acetic acid in water and acetonitrile for the gradient. A single ion peak which absorbed at 240 nM was observed; analysis of this peak in the positive mode indicated maximum mass per charge of approximately 1760 daltons.

APF was purified to homogenity from urine specimens of patients with IC or from culture medium of bladder epithelial cells from patients with IC. It is possible that APF is produced by cells in the kidneys, ureter, or other cells in the uro-kidney tract. If one was so inclined, one skilled in the art would be to purify APF from cells from the kidney, ureter, or other cells from the uro-kidney tract or from fluids obtained from the kidney, ureter, or other cells in the uro-kidney tract using the teachings contained in this application.

6.3 APF and its Functional equivalents, Agonists and Antagonists

The invention also encompasses active functional equivalents or fragments of APF of the invention.

The present invention in various embodiments encompasses novel APF compounds, anti-APF compounds, and active metabolic breakdown products of the APF compounds and anti-APF compounds.

The antiproliferative activity of APF functional equivalents can be confirmed using the assays described herein, or other antiproliferative assays known in the art, to determine whether the functional equivalent retains some or all of antiproliferative activity of APF.

The functional equivalents of the therapeutic compounds include derivatives. Derivatives of therapeutic compounds may be prepared to facilitate chemical and/or physical characteristics desirable for pharmaceutical formulation and/or administration. Examples of such characteristics include improved hydrophilicity, lipophilicity or amphiphilicity characteristics, improved absorption, biological half-life, and/or shelf-life characteristics, etc. Moieties used to derivatize the therapeutic compounds may also be selected to decrease toxicity, eliminate or attenuate any undesirable side effect of the molecule. Examples of moieties capable of mediating such effects are known in the art; examples are disclosed in Remington's Pharmaceutical Sciences (1980). Procedures for coupling such moieties to polypeptide drugs are well known in the art.

APF compounds can be used to diagnose the presence of antibody to APF in tissue or urine or for testing drugs which are suspected of inhibiting APF function. Since APF was shown to inhibit growth of T24 bladder carcinoma cells by ³H-thymidine assay, APF compounds can be provided to a subject for treatment of bladder cancer or other conditions associated with increased cell proliferation.

An “antagonist” of APF is a compound which inhibits the function of APF. Such antagonists can be immunoglobulins (such as, for example, monoclonal or polyclonal antibodies, or active fragments of such antibodies). The antagonists of the present invention also include non-immunoglobulin compounds (such as polypeptides, organic compounds, etc.) Such antagonists may, for example, be identified using high throughput screening of libraries of candidate pharmaceutical compounds for the ability to bind to APF, to its receptor, or to an APF antibody. This binding activity is a measure of the compound's ability to inhibit the activity of APF. Compounds for screening may, for example, be obtained using standard combinatorial chemistry techniques and other high throughput synthesis methods.

Polyclonal antibodies capable of binding to APF can be prepared by immunizing a mammal with a preparation of APF or functional equivalent of APF, such as an APF fragment or a fusion protein comprising an APF compound. Methods for accomplishing such immunizations are well known in the art. Monoclonal antibodies (or fragments thereof) can also be employed to assay for the presence (or amount) or APF in a particular biological sample. Such antibodies can be produced by immunizing splenocytes with activated APF, e.g., by modifying the procedures of Kohler et al.¹¹

In addition to the above methods, antibodies capable of binding to the receptor for APF may be produced in a two-step procedure through the use of anti-idiotypic antibodies, using antibodies to APF as antigens. In accordance with this method, antibodies capable of binding to APF are used to immunize an animal.

The splenocytes of such an animal are then used to produce hybridoma cells, and the hybridoma cells are screened to identify clones that produce antibody whose ability to bind to anti-APF antibodies can be specifically blocked by APF protein. Such antibodies comprise anti-idiotypic antibodies to the anti-APF antibody, and are useful, for example, to immunize an animal to induce the formation of antibodies capable of binding to APF. Anti-idiotypic antibodies, or other agents which mimic APF can also be used as antitumor factors.

As an alternative to, or in addition to, administering APF (or a functional equivalent of APF) to a subject, the efficacy of APF in a subject can be increased, for example, by administering an APF agonist to a subject. Such APF agonists represent an aspect of the invention. The term “agonist” is used broadly to include any compound that is capable of mimicking or improving the efficacy of APF. Examples of such agonists include agents which promotes the synthesis of APF by the subject. Agonists can be used to induce APF production in normal cells for testing drugs and treatments and for diagnostic purposes. Additionally, anti-idiotypic antibodies, or analogs of APF, or agents that mimic APF activity, or a combination of any of the above can are included as APF agonists of the invention.

APF may be obtained synthetically, through the use of recombinant DNA technology, peptide synthesis, or by proteolysis. The therapeutic advantages of such agents may be augmented through the combined administration of several agents.

The invention also includes functional equivalents of APF that lack one, two, or more amino acid residues, or which contain altered amino acid residues, so long as such derivatives exhibit the capacity to influence cell proliferation. For example, in one aspect, APF is truncated at either or both termini by 1, 2, 3, 4 or 5 amino acids.

The compounds of the present invention are preferably provided in a form which is substantially free of natural contaminants. Such preparations are substantially free of materials with which APF is normally and naturally found. For example, APF has been separated from other urine components to provide a composition in which a single peak is evident at 215 nm following HPLC in the fraction that has antiproliferative activity, the molecular weight is between 1700 and 2500 daltons, the activity is heat stable, the pI is about 1.38-3.5, and the composition exhibits an ability to inhibit the proliferation of several different cell types in vitro, including normal bladder epithelial cells and bladder carcinoma cells, as determined by inhibition of ³H-thymidine or bromodeoxyuridine incorporation.

6.4 Methods for Preparing APF

APF may be obtained from natural sources of APF, by culturing cells that produce APF or by using synthetic techniques. For example, APF may be obtained by inducing the production of APF from a human or animal cell, either in vivo or in vitro. Similarly, APF compounds be manufactured using recombinant cells or organisms (e.g., yeast, bacteria, fungi, animal, plant), genetically engineered to produce a specific APF compound. APF is also suitably obtained using synthetic methods, such as, the Merrifield method for synthesizing polypeptides.

APF can be isolated and/or partially purified from sources of APF, such as IC urine or cell cultures producing APF. For example, the inventors have isolated APF from IC urine as well as the medium of bladder epithelial cells isolated from IC patients.

APF compounds can be isolated and/or partially purified using conventional techniques, such as affinity chromatography. For example, antibodies prepared against APF compounds can be used to prepare an affinity chromatography column that can be used to purify the APF compounds by well-known techniques.¹² Antibodies, either polyclonal or, preferably, monoclonal, with specificity for the APF compounds can be generated by the methods described herein or by other methods known in the art.¹³

Fractions of an APF source can be assayed for the presence of APF using a monoclonal antibody specific for APF. The assay can be performed by known methods. For example, an immunoradiometric assay (IRMA) can be used.¹⁴ Briefly, the IRMA assay is performed by adsorbing an antibody against APF onto the surface of wells of a microtiter plate by incubation in a coating buffer (0.2 M sodium bicarbonate, pH 9.5) overnight at 4° C. The residual non-specific binding sites are blocked by the addition of a 1% bovine serum albumin solution (with 0.1% sodium azide) to the wells for 3 hours at room temperature, and the wells of the microtiter plate are then washed with deionized water. An aliquot of the fraction in assay buffer (0.01 M sodium phosphate, 0.15 M NaCl, 0.01 M EDTA, 0.1% sodium azide, 0.1% bovine γ-globulin, pH 7.4) is incubated in the wells for 24 hours at room temperature. The sample is then removed and the wells washed with deionized water. A solution of a second antibody specific for APF, which antibody has been iodinated with I¹²⁵, (approximately 40,000 cpm/well) is incubated in the wells for 24 hours at room temperature. The iodinated antibody solution is removed and the wells washed five times with deionized water. The level of radioactivity in each well is then determined in a scintillation counter which can measure γ-irradiation.

APF compounds can be obtained by recombinant expression techniques.¹⁵ Examples of recombinant expression systems that may be suitably employed in the production of APF compounds include prokaryotic cell systems, eukaryotic cell systems and artificial expression systems.

An expression vector for expressing a nucleic acid sequence encoding an APF compound can be introduced into a cell for expression of an APF compound. In a preferred embodiment, the nucleic acid is DNA. The vector can remain episomal or become chromosomally integrated, as long as it can be transcribed in the host cell to produce the desired RNA. Vectors can be constructed by standard recombinant DNA technology methods. Vectors can be plasmid, viral, or others known in the art, used for replication and expression in, eukaryotic or prokaryotic cells.

For prokaryotic production, any expression vector that is functional in the selected prokaryotic host cell may be used, provided that the vector contains all of the necessary nucleic acid components or elements to ensure expression of an APF compound. Typically, the vector will contain a promoter, an origin of replication element, a transcriptional termination element, a ribosome binding site element, a polylinker region for inserting the nucleic acid encoding the polypeptide to be expressed, and a selectable marker element.

The promoter may be homologous (i.e., from the same prokaryotic species and/or strain as the host cell), heterologous (i.e., from a source other than the prokaryotic host cell species or strain), or synthetic. As such, the source of the promoter may be any unicellular prokaryotic or eukaryotic organism, any vertebrate or invertebrate organism, or any plant, provided that the promoter is functional in, and can be regulated by, the host cell. The more preferred promoters of this invention are inducible promoters, such as those of bacteriophage lambda origin, i.e., lambda promoters, the T5 promoter or the T7 promoter, bacterial promoters such as lac, tac (a composite of the trp and lac promoters), trp, and tna.

The promoter nucleic acid sequences useful in this invention may be obtained by any of several methods well known in the art. Typically, promoters useful herein will have been previously identified by mapping and/or by restriction endonuclease digestion and can thus be isolated from the proper tissue source using the appropriate restriction endonucleases. In some cases, the promoter may have been sequenced. For those promoters whose DNA sequence is known, the promoter may be synthesized using the methods described above for nucleic acid synthesis or cloning. Where all or only portions of the promoter sequence are known, the promoter may be obtained using PCR and/or by screening a genomic library with suitable oligonucleotide and/or promoter sequence fragments from the same or another species. Once isolated, the promoter may optionally be sequenced and prepared synthetically.

Where the promoter sequence is not known, a fragment of DNA containing the promoter may be isolated from a larger piece of DNA that may contain, for example, a coding sequence or even another gene or genes. Isolation may be accomplished by restriction endonuclease digestion using one or more carefully selected enzymes to isolate the proper DNA fragment. After digestion, the desired fragment may be isolated by agarose gel purification, QIAGEN^{™} column or other methods known to the skilled artisan. Selection of suitable enzymes to accomplish this purpose will be readily apparent to one of ordinary skill in the art.

An origin of replication is typically a component of commercially available prokaryotic expression vectors. The origin of replication aids in the amplification of the vector in a host cell. Amplification of the vector to a desirable copy number (e.g., a number which results in maximum production of an APF compound and effective maintenance of the plasmid in the cell culture) can, in some cases, be important for optimal expression of an APF compound. If the vector of choice does not contain an origin of replication site, one may be chemically synthesized based on a known sequence, and ligated into the vector.

A transcription termination element is typically located 3′ to the end of the polypeptide coding sequence and serves to terminate transcription of the polypeptide. Usually, the transcription termination element in prokaryotic cells is a G-C rich fragment followed by a poly T sequence. While the element is easily cloned from a library or even purchased commercially as part of a vector, it can also be readily synthesized using methods for nucleic acid synthesis such as those described above.

Selectable marker genes encode proteins necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; (c) supply critical nutrients not available from complex media; or (d) result in fluorescence or other observable qualities. Preferred selectable markers are the kanamycin resistance gene, the ampicillin resistance gene, and the tetracycline resistance gene.

A ribosome binding site element may also be present. This element, commonly called the Shine-Dalgarno sequence, facilitates translation initiation of a mRNA. The element is typically located 3′ to the promoter and 5′ to the coding sequence of the polypeptide to be synthesized. The Shine-Dalgarno sequence is varied but is typically a polypurine (i.e., having a high A-G content). Many Shine-Delgarno sequences have been identified, each of which can be readily synthesized using methods set forth above.

All of the elements set forth above, as well as others useful in this invention, are well known to the skilled artisan and are described, for example, in Sambrook et al.,¹⁶ Berger et al.¹⁷ and other references.¹⁸

For eukaryotic expression, any promoter known to be effective in the cells in which the vector will be expressed can be used to initiate expression of an APF compound. Suitable promoters may be inducible or constitutive. Examples of suitable eukaryotic promoters include the SV40 early promoter region,¹⁹ the promoter contained in the 3 [symbol] long terminal repeat of Rous sarcoma virus,²⁰ the HSV-1 (herpes simplex virus-1) thymidine kinase promoter,²¹ the regulatory sequences of the metallothionein gene,²² etc., as well as the following animal transcriptional control regions, which exhibit tissue specificity and have been utilized in transgenic animals: elastase I gene control region (active in pancreatic acinar cells);²³ insulin gene control region (active in pancreatic beta cells),²⁴ immunoglobulin gene control region (active in lymphoid cells),²⁵ mouse mammary tumor virus control region (active in testicular, breast, lymphoid and mast cells),²⁶ albumin gene control region (active in liver cells),²⁷ alpha-fetoprotein gene control region (active in liver cells),²⁸ alpha 1-antitrypsin gene control region (active in liver cells),²⁹ beta-globin gene control region (active in erythroid cells),³⁰ myelin basic protein gene control region (active in oligodendrocyte cells in the brain),³¹ myosin light chain-2 gene control region (active in skeletal muscle cells),³² and gonadotropin releasing hormone gene control region (active in cells of the hypothalamus).³³

APF compounds can be prepared for secretion using recombinant DNA technology. This may be accomplished by creating a nucleic acid construct wherein the DNA encoding an APF compound is attached at its 5′ end to a naturally occurring or synthetic DNA sequence encoding a signal peptide. For secretion, the signal peptide sequence selected must be one that is recognized by, and therefore capable of being processed by, the host cell into which this construct is to be inserted and expressed. Thus, for example, a signal peptide obtained from a naturally secreted bacterial polypeptide can be attached to a polypeptide from a source such as human tissue thereby creating a hybrid precursor polypeptide that can be synthesized in, and secreted from, those bacterial (and other prokaryotic) cell species that recognize and are able to process the signal peptide. The hybrid construct can be introduced into the host cell to provide the host cell with the capability of manufacturing and secreting an APF compound.

In one aspect of the invention, a mammal is genetically modified to produce an APF compound in its milk. Techniques for performing such genetic modifications are described in U.S. Pat. No. 6,013,857, issued Jan. 11, 2000, for “Transgenic Bovines and Milk from Transgenic Bovines.” The genome of the transgenic animal is modified to comprise a transgene comprising a DNA sequence encoding an APF compound operably linked to a mammary gland promoter. Expression of the DNA sequence results in the production of an APF compound in the milk. An APF compound may then be isolated from milk obtained from the transgenic mammal (e.g., using a column comprising an antibody which binds to an APF compound). The transgenic mammal is preferably a bovine species.

Segments of the APF compounds may also be prepared using the foregoing recombinant techniques, which may then be modified, for example, to introduce branching or to derivatize the peptides using standard synthetic techniques.

6.5 Anti-APF Antibodies

The invention also includes monoclonal and polyclonal antibodies having binding affinity for one or more of the APF compounds of the invention. The term “antibodies” as used herein is broadly construed to include (1) monoclonal and polyclonal antibodies which bind to one or more APF compounds of the invention, as well as humanized analogs of such antibodies and active fragments of such antibodies which bind to one or more of the APF compounds, and (2) antibodies which bind to the variable regions of the foregoing antibodies, humanized analogs and active fragments.

The antibodies can be manufactured by a wide variety of known methods. As an example, antibodies may be produced by immunizing a host animal by injection with an APF compound. Examples of suitable animals include goats, sheep, donkeys, horses, hamsters, chickens, rabbits, mice, rats, etc. Antibody production may be performed according to a variety of methods known in the art. In a preferred embodiment, antibodies are raised against an APF compound fused to a carrier protein, such as keyhole limpet hemocyanin (KLH), bovine serum albumin (BSA) or ovalbumin (OVA). The antibodies may, for example, be obtained from tissue culture supernatants and/or cell lysates, ascite fluid, serum, plasma and/or whole blood.

Once obtained, the antibodies may be cleaved to provide F(Ab)₂ and/or F(AB) fragments while still maintaining the activity of the uncleaved antibodies. Antibodies can be immobilized on resins, added in solution or coated on other solid support surfaces.

Antibodies of the invention can be used in a variety of assays, for example, enzyme linked immunosorbent assay (ELISA), Westem blot, and immuno-PCR assays, and are also useful in solution or solid phase affinity quantification/qualification. In a preferred embodiment the assay is an ELISA.

Various adjuvants may be employed in the production of antibodies of the invention, to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies, any technique that provides for the production of antibody molecules by continuous cell lines in culture may be used. For example, the hybridoma technique originally developed by Kohler and Milstein,³⁴ as well as the trioma technique, the human B-cell hybridoma technique,³⁵ and the EBV-hybridoma technique to produce human monoclonal antibodies.³⁶ Monoclonal cells lines can then be screened for binding to the particular APF compounds using the purified species in any type of immunoassay available in the art.³⁷

It will be recognized by one of skill in the art that the antibodies of the invention have a wide range of uses, e.g., in the isolation, qualitative characterization, and quantification of APF; isolation, qualitative characterization, and quantification of natural and synthetic functional equivalents of APF; as well as the isolation, qualitative characterization, and quantification of immunologically cross-reactive materials derived from other biological sources e.g., primate, rodent, ovine, porcine and ovoid species.

The antibodies of the invention are useful for monitoring urine levels of the APF compounds by methods known in the art. Antibodies to the APF compounds are also useful for tracking delivery of the APF compounds. The antibodies of the invention can be linked to a suitable tag to allow visualization of sites where the antibodies have accumulated, e.g., where they have bound to the APF compounds or other polypeptides. In one aspect of the invention, tagged antibodies are administered to a subject to identify the site of accumulation or expression of the APF compounds.

Analysis of compounds using the antibodies of the invention may be accomplished in biological fluids and physiological buffers. Moreover, the antibodies may be immobilized by attachment to a suitable support structure according to known methods for isolation and purification of the APF compounds.

The antibodies of the invention are also useful for in vivo therapeutic administration and manipulation of naturally occurring levels of APF, as well as in vitro analysis of production levels, location and species, study of pharmacokineticaly relevant levels of APF compounds of the invention, half-life and in vivo distribution, degradation, manipulation and sites of production, depot and action. In this regard, the antibodies of the invention may be bound to agents having affinity for target tissues. Conversely, the antibodies themselves are usefully employed in the targeting of therapeutics to APF-reactive sites, blocking of activity in vitro or in vivo, modification of solution kinetics, and a wide variety of other uses that would be apparent to one of skill in the art.

In one aspect of the invention, one or more antibodies of the invention is administered to a subject to block the activity of APF.

Moreover, the antibodies of the invention are useful for identifying functional domains, folding patterns, sequence and in investigating the effects of in vivo/in vitro post-translational modifications on solubility of APF. The antibodies of the invention also find a variety of uses in the investigation of solubility, physio-chemical, biological, stability, degradation and structural characteristics of the APF compounds.

The antibodies of the invention are useful in the detection and purification of APF compounds. The antibodies of the invention may be linked to a variety of accessory molecules to aid in purification, analysis, assay characteristics, as well as to improve targeting, tracking and biological half-life, depoting, location, and cofactor use. For example, antibodies can be tagged by known methods with markers, such as with radioactive markers, fluorescent markers, chemiluminescent markers or affinity tags, such as biotin.

The antibodies of the invention are useful in qualitative and quantitative assays for APF compounds and also for immunologically cross-reactive materials derived from other biological sources i.e., primates, rodents, ovine, porcine and ovoid species. Examples of suitable sources include urine, serum, plasma and whole-blood collected from suitably qualified donors, tissue sections, biopsy samples bacterial/tissue culture supernantants/lysates prepared from transfected/transformed cell cultures, and partially purified materials derived from these sources.

The described antibodies can be utilized in the purification of APF compounds from synthetic and natural sources. Examples of suitable starting materials include, but are not limited to urine, serum, plasma and whole-blood collected from suitably qualified donors, tissue sections, biopsy samples, bacterial/tissue culture supernantants/lysates prepared from cell cultures (particularly cultures originating from cells of IC patients), and partially purified materials derived from these sources.

Antibodies to an APF compound can be immobilized to a support, such as a silica or sepharose bead for use in isolating the APF compound. Antibodies may be positioned in a manufacturing system (e.g., downstream from an incubator where an APF compound is being produced by recombinant organisms) for isolating the APF compound. Examples of suitable production methods include chemical synthesis, expression in a suitable recombinant expression vector/ host cell culture systems, and isolation from urine, serum or plasma (e.g., urine, serum or plasma produced by an IC patient or by a recombinant organism).

The antibodies may be manipulated according to various techniques known in the art to achieve alteration of soluble material concentrations, complexing material so as to reduce or enhance half-life in solute, complexing to neutralize or otherwise modify activity against target entities, modification of biological target.

The antibodies of the invention are also useful for the production of anti-idiotype antibodies, which will mimic the therapeutic activities of the APF compounds described herein. In this embodiment, the antibodies are used as immunogens in suitable animal species (e.g., rat, mouse or rabbit) to produce an anti-idiotype antibody that mimics the activity of an APF compound. A preferred immunogen is a monoclonal antibody that, upon incubation with an APF compound, inactivates one or more of the therapeutic activities of the APF compound.

6.6 Diagnostic Assays, Methods and Kits

The invention provides methods for diagnosing IC by analyzing a biological sample from a subject for the presence of APF, and optionally for the amount of APF, in the sample. A positive result is indicated by the presence of APF or by the presence of an elevated amount of APF.

The biological sample employed in the diagnostic method of the invention may, for example, be urine; an subfraction of urine; a tissue sample; or other biological sample.

The samples may be analyzed by any method for detecting the presence, and optionally the amount, of a specific polypeptide in the biological sample. A wide variety of such methods are known in the art.

Moreover, in situ assay of cells, or organ or tissue sections may also be employed in the diagnostic methods of the invention.

For example, the level of APF present in the urine of a suspected IC patient can be detected by incubating primary normal adult human bladder epithelial cells (HBE) with the urine (preferably whole urine). Proliferation of the HBE cells is then measured, e.g., by determining the level of inhibition of ³H-thymidine or BrdU incorporation in the cells. This measurement is compared to the level of proliferation of HBE cells incubated with control urine, i.e., urine from age-, race- and sex-matched control persons without urologic disease.

Alternatively, APF can be detected and/or quantified using an antibody-based assay. A monoclonal antibody-based assay is preferred. Assays using fragments of polyclonal or monoclonal antibodies are also known in the art. In immunoassays, the antibodies may be utilized in liquid phase or bound to a solid-phase carrier. The antibodies may be labeled using any of a variety of labels and methods of labeling. Examples include enzyme labels, radioisotopic labels, non-radioactive isotopic labels, fluorescent labels, and chemiluminescent labels.

Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, acetylcholine esterase, etc.

Examples of suitable radioisotopic labels include ³H, ¹¹¹In, ¹²⁵I; ³²P, ³⁵S, ¹⁴C, ⁵⁸Co, ⁵⁹Fe, ⁷⁵Se, ¹⁵²Eu, ⁹⁰Y, ⁶⁷Cu ²¹Ci, ²¹¹ At, ²¹²Pb, ⁴⁷Sc, ¹⁰⁹Pd, etc.

Examples of suitable non-radioactive isotopic labels include ¹⁵⁷Gd, ⁵⁵Mn, ¹⁶²Dy, ⁵²Tr, ⁴⁶Fe, etc.

Examples of suitable fluorescent labels include ¹⁵²Eu label, fluorescein label, isothiocyanate label, rhodamine label, phycoerythrin label, phycocyanin label, allophycocyanin label, fluorescamine label, etc.

Examples of suitable chemiluminescent labels include luminal label, isoluminal label, aromatic acridinium ester label, imidazole label, acridinium salt label, oxalate ester label, luciferin label, luciferase label, and the like.

Other suitable labels are known to those of skill in the art.

The binding of labels to antibodies or fragments thereof can be accomplished using standard techniques. Examples of suitable techniques are described by Kennedy et al.³⁸ by Schurs et al.³⁹ Coupling techniques described in Schurs et al. include the glutaraldehyde method, the periodate method, the dimaleimide method, and others, all of which are incorporated by reference herein.

The detection of the antibodies (or fragments of antibodies) of the present invention can be improved through the use of carriers. Well-known carriers include glass, polystyrene, polypropylene, polyethylene, dextran, nylon, amylases, natural and modified celluloses, polyacrylamides, agaroses, and magnetite. The carrier may be soluble or insoluble. The support material may have virtually any possible structural configuration, so long as the coupled molecule is capable of binding to APF. Thus, the support configuration may be spherical, as in a bead, or cylindrical, as in the inside surface of a test tube, or the external surface of a rod. Alternatively, the surface may be flat such as a sheet, test strip, etc. Many suitable carriers for binding monoclonal antibodies are known in the art, and others may be readily ascertained by routine experimentation.

The antibodies, or fragments of antibodies of APF may be used to quantitatively or qualitatively detect the presence of activated APF. Such detection may be accomplished using any of a variety of immunoassays known to persons of ordinary skill in the art such as radioimmunoassays, immunometic assays, etc. Using standard methodology well known in the art, a diagnostic assay can be constructed by coating on a surface (i.e. a solid support) for example, a microtitration plate or a membrane (e.g. nitrocellulose membrane), antibodies specific for APF or a portion of APF, and contacting it with a patient sample, such as urine from a person suspected of having IC. The presence of a resulting complex formed between APF in the urine and antibodies specific therefor can be detected by any of the known detection methods common in the art such as fluorescent antibody spectroscopy or colorimetry. A good description of a radioimmunoassay may be found in Laboratory Techniques and Biochemistry in Molecular Biology,⁴⁰ incorporated herein by reference. Sandwich assays are described by Wide.⁴¹

The invention also provides a diagnostic kit for use in diagnosing interstitial cystitis. The kit may suitably comprise any of the materials described herein for use in such diagnosis. For example, in a preferred embodiment, the kit comprises: (1) a means for measuring levels of APF in a sample (e.g., a sample of urine); and (2) a means for indicating whether the measurement in (1) falls in a range associated with interstitial cystitis. The means of (2) can, for example, comprise written instructions, color coded results, and the like.

6.7 Treatment of Conditions Associated with Excessive Cellular Proliferation

In the treatment of proliferative disorders, the isolated APF may be administered to a subject, either alone or as a component of a pharmaceutical composition.

The APF is suitably administered to inhibit epithelial cell production in a subject in need of such inhibition. The subject is preferably a human subject. The method of administration may comprise any method by which the epithelial cells are contacted with APF. The APF may be administered in purified form or as a component of a pharmaceutical composition. The APF is either synthetic or natural, and is purified in a manner which renders it pharmaceutically acceptable. Pharmaceutically acceptable purity is achieved by removing sufficient contaminating substances (i.e., substances remaining from the source of the APF, such as substances from the culturing of bladder cells or from IC urine) to ensure that the risk of adverse effects caused by any remaining contaminating substances is outweighed by the therapeutic benefit of the APF to the subject.

The APF of the invention is particularly suitable for treatment hyperproliferative disorders affecting the uroepithelium (also referred to in various texts as “transitional epithelium” or “urothelium”), which lines the urinary bladder, the ureter and the upper part of the urethra.

The APF and functional equivalents of the invention may be administered to downregulate HB-EGF production. Such methods generally comprise contacting an HB-EGF-producing cell with the APF or functional equivalent or with a composition comprising the APF or functional equivalent. The APF is preferably isolated from a composition comprising materials secreted from bladder cells of an IC patient. For example, APF may be isolated from an in vitro culture of bladder cells from an IC patient. Alternatively, the IC may be isolated from IC urine or produced synthetically.

A preferred group of cells which may be targeted for treatment using the APF compounds of the invention includes: hepatocytes, keratinocytes, gastric epithelial cells, kidney epithelial cells, and bladder epithelial cells.

The APF compounds of the invention are also useful for regulating production of HB-EGF, EGF, IGF1 and IGFBP3 in a subject. These factors may be regulated by administering the APF compounds of the invention. The production of HB-EGF, EGF, IGF1 and IGFBP3 are preferably by cells of the uro-kidney system, preferably epithelial cells of the uro-kidney tract, most preferably uroepithelium.

APF, or agents which increase the level of APF, or agonists of APF, may be used in the therapy of any disease associated with an increase in cell proliferation wherein APF is capable of decreasing or inhibiting such proliferation, e.g. bladder carcinoma.

The dosage of the APF compounds administered according to the foregoing methods will vary depending upon such factors as the patient's age, weight, height, sex, general medical condition, previous medical history, etc. Where the therapeutic agent is an antibody, it is desirable to provide the recipient with a dosage of antibody which is in the range of from about 1 pg/kg to 10 mg/kg (body weight of patient), although a lower or higher dosage may be administered.

Similarly, agents which are capable of inducing the expression, production, stability or function of APF, are intended to be provided to recipient subjects in an amount sufficient to effect the induction of APF and to improve the condition of the subject. An amount is said to be sufficient to “effect” the induction of APF if the dosage, route of administration, etc. of the agent are sufficient to positively influence such a response.

The antiproliferative activity of the APF compounds of the invention is consistent with the use of the APF compounds in cancer therapy. The APF compounds of the invention are useful as a monotherapy or in combination with other therapies, such as including radiation or chemotherapy. In one aspect of the invention an APF compound is administered in conjunction with another chemotherapy (e.g., treatment with tamoxifen, adriamycin, etoposide, bleomycin, vincristine, vinblastine, doxorubicin, paclitaxel and/or docetaxal). Examples of other suitable antineoplastics for use in combination therapies with the APF compounds of the invention include: adrenocorticotrophic hormones (e.g., corticotropin); antibiotic antineoplastics (e.g., plicamycin); miscellaneous antineoplastics (e.g., gallium nitrate); bone resorption suppression agents (e.g., etidronate disodium and pamidronate disodium); and glucocorticoids (e.g., adrenal cortex; betamethasone; budesonide; cloprednol; cortisone acetate; cortivazol; deflazacort; dexamethasone; fluprednisolone; hydrocortisone; hydrocortisone acetate; hydrocortisone cypionate; hydrocortisone hemisuccinate; hydrocortisone sod phosphate; hydrocortisone sod succinate; meprednisone; methylprednisolone; methylprednisolone acetate; methylprednisolone hemisucc; methylprednisolone sod succ; paramethasone acetate; prednisolone; prednisolone; prednisolone acetate; prednisolone hemisuccinate; prednisolone Na Met-Sul-Benz; prednisolone sod phosphate; prednisolone sod succinate; prednisone; prednylidene; prednylidene).

The APF compounds may be usefully employed in the treatment of leukemias. Specific leukemias which may be treated using an APF compound of the invention include, for example, acute leukemia, such as acute lymphocytic leukemia and acute myelocytic leukemias (e.g., myeloblastic, promyelocytic, myelomonocytic, monocytic and erythroleukemia), and chronic leukemia (e.g., chronic myelocytic (granulocytic) leukemia and chronic lymphocytic leukemia).

Additionally, the APF compounds may be usefully employed in the treatment of polycythemia vera, lymphoma (e.g., Hodgkin's disease and non-Hodgkin's disease), multiple myeloma, Waldenstrom's macroglobulinemia, and heavy chain disease.

Moreover, the APF compounds may be useful in the treatment of solid tumors, including sarcomas and carcinomas, such as fibrosarcoma, myxosarcoma, liposarcoma, chondrosarcoma, osteogenic sarcoma, chordoma, angiosarcoma, endotheliosarcoma, lymphangiosarcoma, Kaposi's sarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms′ tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and virally-induced cancers.

In a preferred aspect of the invention, an APF compound is administered to treat a bladder cancer. For example, the bladder cancer may comprise a transitional cell carcinoma, a squamous cell tumor, or an adenocarcinoma. The most preferred cancer for treatment using an APF compound of the invention is a transitional cell carcinoma.

Where the APF compound is used in the treatment of a bladder carcinoma, it may be administered as a monotherapy, or in conjunction with other chemotherapy agents, as a component of a multi-drug treatment regimen. Moreover, the APF compound may be administered in conjunction with other types of treatment, such as radiotherapy. Other chemotherapy agents particularly suited for use in combination therapies with the APF compounds of the invention include: methotrexate, vinblastine, doxorubicin, cisplatin, and granulocyte colony stimulating factor (G-CSF).⁴²

The invention provides a method for screening an APF compound of the invention for anti-cancer activity. The method comprises assaying an APF compound of the invention for the ability to inhibit the survival or proliferation of malignant cells.

In one embodiment, the preparation is screened by a method comprising: (1) contacting malignant cells with an APF compound of the invention; (2) measuring the survival or proliferation of malignant cells; and (3) comparing the survival or proliferation of the cells contacted with an APF compound of the invention with the survival or proliferation of cells not so contacted (e.g., cells contacted with a control). A lower level of survival or proliferation in the contacted cells indicates that the preparation has anti-cancer activity. Examples of suitable cells are those which are derived from or display characteristics associated with a malignant disorder.

Cells may also be screened for the ability of an APF compound of the invention to convert cells having an abnormal phenotype to a more normal cell phenotype. For example, suitable cells may include pre-neoplastic or pre-malignant cells. A more normal phenotype in the contacted cells indicates that the preparation has anti-cancer activity.

In vitro assays can be used to determine whether administration of a specific APF compound of the invention is indicated in a specific subject. For example, the invention provides in vitro cell culture assays. A tissue sample obtained from a subject is grown in culture and exposed to or otherwise administered an APF compound of the invention. The effect of an APF compound of the invention upon the tissue sample is observed. In one embodiment, where the subject has a malignancy, a sample of cells from the malignancy is plated out or grown in culture. The cells are then exposed to an APF compound of the invention. An APF compound which inhibits survival or growth of the malignant cells is selected for therapeutic use in vivo.

Alternatively, in vitro assays can be carried out using a cell line, rather than a cell sample derived from the specific subject to be treated. The cell line is preferably derived from or displays characteristic(s) associated with the malignant, neoplastic or pre-neoplastic disorder desired to be treated or prevented, or is derived from the cell type upon which an effect is desired, according to the invention.

Many assays standard in the art can be used to assess such survival and/or growth. For example, cell proliferation can be assayed by measuring ³H-thymidine incorporation, by direct cell count, by detecting changes in transcriptional activity of known genes such as proto-oncogenes (e.g., fos, myc) or cell cycle markers. Cell viability can be assessed by trypan blue staining. Cell differentiation can be assessed visually based on changes in morphology, etc.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. The APF compounds can also be assessed in clinical trials in human subjects presenting with a specific neoplastic disease.

6.8 Treatment of Conditions Associated with APF

In another aspect, the invention provides a method for treating a condition associated with the presence of APF or the presence of elevated APF. In this method, a subject in need of such treatment is administered a therapeutically effective amount of an agent, which inhibits APF or which otherwise overcomes the effects of APF. For example, the therapeutic agent may suitably comprise an anti-APF antibody or a fragment of an anti-APF antibody, which binds to the APF.

Agents which decrease the level of APF (i.e. in a human or an animal) or inhibit APF activity may be used in the therapy of any disease associated with the presence of APF.

The dosage of the anti-APF agents will vary depending upon such factors as the patient's age, weight, height, sex, general medical condition, previous medical history, etc. Where the therapeutic agent is an antibody, it is desirable to provide the recipient with a dosage of antibody which is in the range of from about 1 pg/kg to 10 mg/kg (body weight of patient), although a lower or higher dosage may be administered.

The antibodies or compounds capable of inhibiting APF, that is inhibiting either the production or activity of APF, are intended to be provided to recipient subjects in an amount sufficient to effect inhibition of APF and to improve the condition of the subject. An amount is said to be sufficient to “effect” the inhibition or induction of APF if the dosage, route of administration, etc. of the agent are sufficient to positively influence such a response.

6.9 Pharmaceutical Compositions and Methods of Administration

The therapeutic compounds of the invention are suitably administered as components of pharmaceutical compositions. The pharmaceutical compositions of the invention generally comprise a therapeutic compound and a pharmaceutically acceptable carrier, where the therapeutic compound is the APF or its functional equivalent or another therapeutic compound described herein.

The invention provides methods of treatment and prevention by administration to a subject in need of such treatment of a therapeutically or prophylactically effective amount of one or more therapeutic compounds of the invention. The subject is preferably an animal, including, but not limited to, animals such as monkeys, cows, pigs, horses, chickens, cats, dogs, etc., and is preferably a mammal, and most preferably human.

Various delivery systems are known and can be used to administer therapeutic compounds of the invention. For example, suitable systems include: encapsulation in liposomes, microparticles and/or microcapsules, recombinant cells capable of expressing the therapeutic compounds; receptor-mediated endocytosis;⁴³ plasmids encoding one or more therapeutic compounds; viral vector delivery systems, etc. The therapeutic compounds can be delivered in a vesicle, in particular a liposome.⁴⁴

Routes of introduction include but are not limited to intradermal, intramuscular, intraperitoneal, intravenous, subcutaneous, intranasal, epidural, and/or oral routes. Intrabladder administration is preferred. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection. An intraventricular catheter may be used to facilitate intraventricular injection, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. For example, local administration may be achieved by topical application, by injection, by means of a catheter, by means of a suppository, or by means of an implant, the implant being of a porous, non-porous, or gelatinous material, including membranes, such as sialastic membranes, or fibers.

In yet another embodiment, an APF compound can be delivered in a controlled release system. A pump may be used as needed.⁴⁵ Polymeric materials may also be employed in a controlled release system, according to methods known in the art.⁴⁶ In yet another embodiment, a controlled release system can be placed in proximity of the therapeutic target, thus requiring only a fraction of the systemic dose.⁴⁷ Other controlled release systems are discussed in the review by Langer.⁴⁸

In a specific embodiment nucleic acids encoding one or more therapeutic compounds of the invention is administered using gene therapy methods.

The term “carrier” refers to a diluent, adjuvant, excipient, or vehicle with which the therapeutic compound is administered to a subject. Examples of suitable pharmaceutical carriers are described in “Remington's Pharmaceutical Sciences” by E. W. Martin. Such compositions will contain a therapeutically effective amount of one or more therapeutic compounds of the invention, preferably in purified form, together with a suitable amount of carrier so as to provide the form for proper administration to the subject. The formulation should suit the mode of administration. In a preferred embodiment, the composition is formulated in accordance with routine procedures as a pharmaceutical composition adapted for intravenous administration to human beings. The compositions may also be formulated for veterinary use.

Examples of suitable pharmaceutical carriers include sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides.

Oral formulation can include standard carriers such as pharmaceutical grades of mannitol, lactose, starch, magnesium stearate, sodium saccharine, cellulose, magnesium carbonate, etc.

Typically, compositions for intravenous administration are solutions in sterile isotonic aqueous buffer. Where necessary, the composition may also include a solubilizing agent and a local anesthetic such as lignocaine to ease pain at the site of the injection.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An APF compound of the invention can be formulated in neutral or salt form. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic compound of the invention that will be effective in the treatment of a particular disorder or condition depends on various factors and can readily be determined by one of skill in the art using standard clinical techniques with reference to the instant disclosure. For example, dosage amounts will depend on the nature of the disorder or condition. In vivo and/or in vitro assays may optionally be employed to help predict optimal dosage ranges. Effective doses may also be extrapolated from dose-response curves derived from the in vitro and in vivo experiments described herein.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The pharmaceutical composition may also be formulated to control the duration of action. Controlled release preparations may be achieved through the use of polymers to complex or absorb the compounds. The controlled delivery may be exercised by selecting appropriate macromolecules (for example polyesters, polyamino acids, polyvinyl, pyrrolidone, ethylenevinylacetate, methylcellulose, carboxymethylcellulose, or protamine sulfate) and the concentration of macromolecules as well as the method of incorporation in order to control release. Another means for controlling the duration of action by controlled release preparations is to incorporate the compounds of the present invention into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacrylate)microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980).

6.10 Other Methods

In addition to the ensuing diagnostic and therapeutic methods, the invention also provides a method for identifying the structure of an APF from urine of subjects with interstitial cystitis, the method comprising analyzing the structure of the APF using known analytic techniques, such as one-dimensional and/or two-dimensional NMR, and mass spectroscopy. The sequence of peptides generated by cleavage with cyanogen bromide and/or other cleavage can be performed.⁴⁹ The N-terminus of APE can also be deblocked by chemical methods for removal of a formyl group. The amino acid content may be determined by various methods known in the art, e.g., ion trap mass spectrometry. The amino acid sequence of the isolated polypeptide can be obtained using standard sequencing techniques known in the art.

Additionally, the invention includes methods for identifying anti-APF compounds by screening compounds for the capacity to inhibit the antiproliferative activity of APF. For example, such screening can be accomplished using combinatorial chemistry techniques and high-throughput screening, by which large numbers of compounds can be tested, preferably in automated fashion, for activity of inhibitors of the APF. The primary goal of high throughput screening is to identify compounds that inhibit the antiproliferative activity of the APF that are active at a fairly low concentration. The lower the concentration at which the compound acts, the more likely that it will exhibit specificity and, as a corollary, the less likely that it will have undesired side effects.⁵⁰

An HTS requires four elements: (1) suitably arrayed compound libraries; (2) an assay method configured for automation; (3) a robotics workstations; (4) a computerized system for handling the data. The 96-well microtiter plate is the standard format for automated assays, although arrays of compounds on chips or on beads are also used and assays can be performed on agar plates or other solid support. Synthesis of combinatorial libraries can be accomplished in microtiter plates, thereby providing addresses for particular compounds generated by a given subset or series of reactions and thus identifying the compound. For further information on how to use HTS for determining anti-APF compounds.⁵¹ APF agonist, i.e., compounds that exhibit the antiproliferative activity of APF, may be identified in like manner.

7. Identification of APF

Having now generally described the invention, the same will be more readily understood through reference to the following examples, which are provided by way of illustration, and are not intended to be limiting to the present invention, unless specified.

### 7.1 Example I

7.1.1 Methods and Materials

The following were used in the performance of the studies described in Example I.

Subjects

IC patients were referred by physicians, the National Institute of Diabetes and Digestive and Kidney Diseases (NIDDK), and the Interstitial Cystitis Association. All patients had previously undergone diagnostic cystoscopy and fulfilled the NIDDK diagnostic criteria for IC [Division of Kidney, Urologic, and Hematologic Diseases (DKUHK) of the National Institute of Diabetes and Digestive and Kidney Diseases (NIDDK)(1989) Am J. Kidney Dis. 13:353]⁵²; urine was collected from these patients at least three months following the most recent known bacterial urinary tract infection and one month following the last antibiotic use. Age-, race- and sex-matched controls were volunteers with no history of IC or other urological disease, or patients undergoing cystoscopy for other urological disease (including benign stricture of the ureteropelvic junction, glomerulosclerosis of the kidney, caliceal diverticulum with stones, ureteral endometriosis, and renal cell carcinoma); each control patient was required to have no symptoms of urinary tract infection or antibiotic use for at least one month. All participants were at least 18 years old and enrolled in accordance with guidelines of the Institutional Review Board at the University of Maryland School of Medicine. The number of IC patients or controls used for each experiment was based on the number for whom a sufficient quantity of urine was available for each experiment. However, each IC patient urine specimen was studied simultaneously with urine from one or two age-, race- and sex-matched controls for each experiment.

In later experiments, IC patients were referred by physicians and the Interstitial Cystitis Association. All patients had previously undergone diagnostic cystoscopy and fulfilled the NIDDK diagnostic criteria for IC. Asymptomatic controls were age-, race-, and sex-matched volunteers with no history of IC or other urological disease. Patients with acute bacterial cystitis were identified by the presence of bacteriuria (>10³ bacteria/ml of a single type of bacterium; 17/20 patients had >10⁵ bacteria/ml) plus pyuria in combination with appropriate symptoms. Vulvovaginitis was diagnosed by physical examination. All participants were at least 18 years old and enrolled in accordance with guidelines of the Institutional Review Boards at the University of Maryland School of Medicine and the University of Pennsylvania at Philadelphia.

Urine Specimens

Urine was collected either by catheterization, as previously described,⁵³ or by the clean catch method in which each patient wiped the labial area with 10% povidone iodine/titratable iodine 1% solution (Clinidine, Guilford, Conn.), then collected a midstream urine into a sterile container. Specimens were initially kept at 4° C., transported to the laboratory within one hour of collection, aliquoted under sterile conditions, and plated directly onto confluent cells for cytotoxicity assays or stored at −80° C. until used.

In later experiments, urine was collected by the clean catch method as described above. Specimens were initially frozen at 20° C., transported to the laboratory on ice, thawed, aliquoted under sterile conditions, and stored at −80° C. until used.

Cell Culture

T24 bladder carcinoma cells (ATCC #4-HTB) (Rockville, Md.) were grown in McCoy's medium containing 10% fetal bovine serum (FBS), 1% antibiotic/antimycotic solution, and 1% glutamine (Sigma, St. Louis, Mo.).

Normal adult human bladder epithelial cells (HBE) were grown from biopsies obtained at autopsy from patients who had no history of bladder disorder [Trifillis,et al. (1993) In vitrol Cell Dev. Biol. 29A:908]⁵⁴. The explanted cells were grown in Eagle's minimal essential medium (MEM) containing 10% heat inactivated FBS, 1% antibiotic/antimycotic solution, 1% glutamine, and 1.0 ug/ml insulin (all from Sigma).

Normal human fetal bladder cells FHS 738 B1 (ATCC #160-HTB) were grown in DMEM containing 1000 mg/L glucose, 10% fetal bovine serum, 1% antibiotic/antimycotic solution, 1% glutamine, and 3.5 ug/ml insulin (all from Sigma). All cells were cultured at 37° C. in a 5% CO₂ atmosphere.

³H-Thymidine Incorporation

HBE cells explanted from bladder tissue or FHS 738 B1 cells were plated at a density of 1×10⁴ cells/well onto 96 well tissue culture plates and incubated at 37° C. overnight (resulting in approximately 60% confluence the following day). The medium was then changed to MEM containing only 1% glutamine and 1% antibiotic/antimycotic solution, and the cells were incubated at 37° C. overnight. On the third day urine specimens from IC patients and control were corrected to pH 7.2 and 300 mOsm, filtered, diluted in MEM (with only glutamine and antibiotics/antimycotics) and applied to the cells. Undiluted IC patient or control urine was uniformly extremely toxic in initial experiments, indicating the need for cell culture medium to support the growth of these cells in vitro. Following 48 hours of incubation at 37° C., the cells were pulsed with 1.0 μCi ³H-thymidine/well (NEN DuPont, Wilmington, Del.) and incubated for another 4 hours at 37° C. Cells were trypsinized, lysed with deionized/distilled water, and insoluble cell contents harvested and methanol-fixed onto glass fiber filter paper using a PHD cell harvester (Cambridge Technology, Inc., Watertown, Mass.); the amount of radioactivity incorporated was determined as counts per minute using a Beckman LS 3801 scintillation counter.

Bromodeoxyuridine (BrdU) Incorporation

HBE cells were cultured in 96 well plates and urine specimens applied as described above for the ³H-thymidine incorporation assay. Following 48 hours of incubation with the urine specimens, the cell medium was removed and cells were incubated with BrdU labeling solution (Boehringer-Mannhiem) for 4 hours at 37° C., according to the manufacturer's directions. This solution was then removed, FIXDENAT solution applied, and the cells incubated at room temperature for 30 minutes. The cells were then further incubated with anti-BrdU-peroxidase-labeled antibody, rinsed 3 times with a washing solution, and developed with a substrate solution. Development was stopped with 1M H₂SO₄ and absorbance read at 450 nm.

Dialysis of Urine Specimens

Urine specimens were dialyzed against PBS at 4° C. overnight, using Spectra/Por Membranes (Spectrum Medical Industries, Houston, Tex.) with pore sizes that allowed removal of substances less than 1,000, less than 10,000 or less than 25,000 daltons. The specimens were then removed and pH adjusted to 7.2, as above. The volume recovered for each specimen following dialysis was 90-100% of the original starting volume.

Trypsinization of Urine

Urine was incubated with 8.25 U/ml trypsin conjugated to agarose beads in Hank's buffer (Sigma) at 37° C. for 2 hours, after which the beads were removed by centrifugation, pH and osmolality of the urine adjusted, and ³H-thymidine incorporation assay performed. Duplicate control specimens were incubated with an equivalent amount of Hank's buffer at 37° C. for 2 hours.

Statistical Analysis

Differences in the number of specimens causing significant inhibition of ³H-thymidine or BrdU incorporation were analyzed by Fisher's exact test (where significant inhibition was defined as a decrease greater than 2 standard deviations from the mean of controls). Comparisons of mean change in ³H-thymidine incorporation caused by undialyzed vs. dialyzed urine specimens were performed using a one-way analysis of variance with Scheffe's test for multiple comparisons (IC patient and control specimens analyzed separately).

In later experiments, comparison of the number of IC patients and controls whose urine inhibited cell proliferation was performed using Fisher's exact test; significant inhibition was defined as a decrease in 3H-thymidine or BrdU incorporation greater than 2 standard deviations from the mean of untreated control cells. A comparison of the mean percent change in ³H-thymidine incorporation for the IC group vs. each of the 3 control groups was also made using a two tailed analysis of covariance with age as the covariate.

7.1.2 Identification of a Putative APF (APF) in Urine from IC Patients

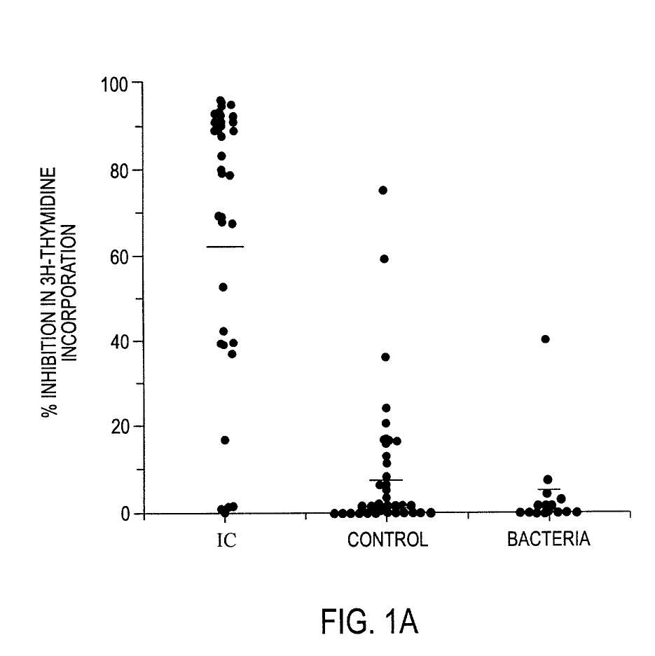

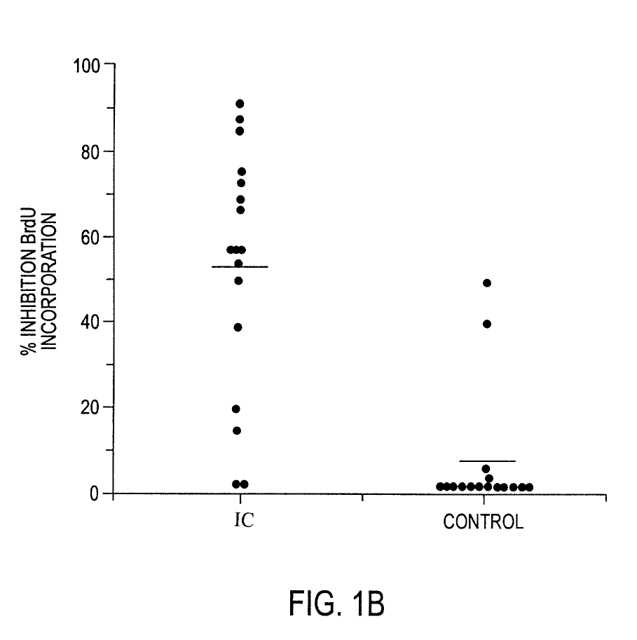

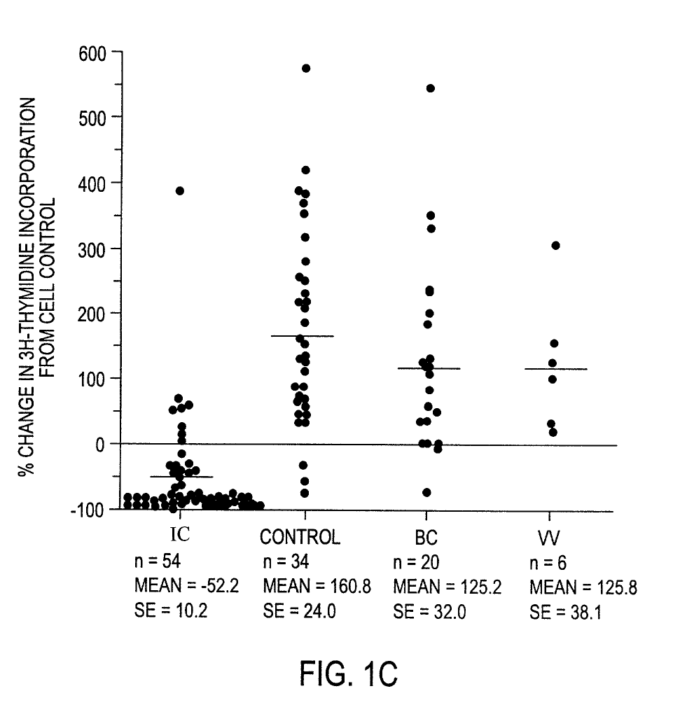

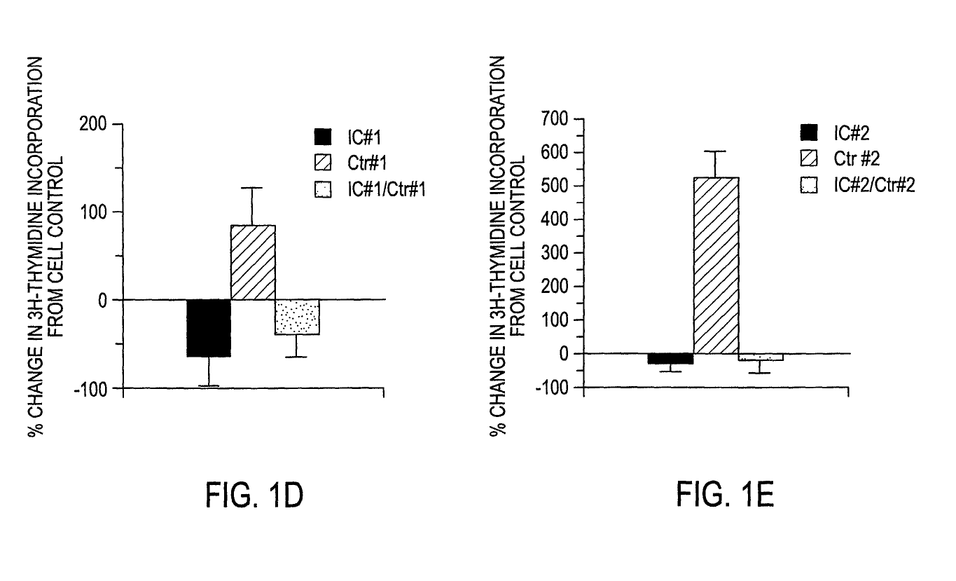

IC patients whose diagnosis was confirmed by cystoscopy at the University of Maryland and who fulfilled criteria established by the NIDDK were studied. Primary normal adult human bladder epithelial (HBE) cells were incubated with whole urine specimens from these IC patients or age-, race- and sex-matched controls. Prior to their addition to the cell culture medium, all specimens were corrected for osmolality (300 mOsm) and pH (7.0). The results indicate that the proliferation of HBE cells is inhibited by urine from IC patients as compared to age-, race- and sex-matched controls without urologic disease and to patients with bacterial cystitis. Specimens from 22 of 29 (76%) IC patients vs. 2 of 33 (6%) controls inhibited HBE cell proliferation significantly as determined by ³H thymidine incorporation in vitro (FIG. 1A; p<0.001, Fisher's exact test analysis; significant inhibition was defined as a decrease greater than 2 standard deviations from the mean of untreated control cells). This finding was confirmed by bromodeoxyuridine incorporation using specimens from a subset of these patients [12 of 16, or 75% of IC patients had significant inhibition vs. 2 of 16, or 12% of controls (FIG. 1B; p=0.001 by Fischer's exact test)].

To determine the reproducibility and specificity of this finding, the initial studies were expanded; to date, the following have been screened for antiproliferative urine activity by ³H-thymidine incorporation:

urine from 54 women with IC (mean age 44.1±2.0 years);

34 asymptomatic control women (mean age 41.0±2.0 years);

20 women with documented bacterial cystitis (mean age 24.3±1.4 years); and

6 women with vulvovaginitis (mean age 37.0±5.6 years);

Specimens from 46 of 54 (85%) IC patients inhibited human bladder epithelial cell proliferation in vitro, as compared to 3 of 34 (9%) asymptomatic controls, 2 of 20 (10%) patients with bacterial cystitis, and 0 of 6 (0%) women with vulvovaginitis. The mean percent change in ³H thymidine incorporation in cells cultured with IC urine was −52.2+/−10.2, as compared to +160.8 +/−24.0 for asymptomatic controls, +125.2 +/−32.0 for bacterial cystitis patients and +125.8+/−38.1 for vulvovaginitis patients (FIG. 1C, p<0.001 for each comparison of the IC group to each of the 3 control groups using a two tailed analysis of covariance with age as the covariate). Each data point is the mean of six samples. Mean value and standard error for the population are indicated for each group.

7.1.3 Demonstration of Diagnostic Utility of APF

The diagnostic utility of APF was demonstrated by determining the sensitivity, specificity, positive predictive value and negative predictive value of a significant decrease in ³H-thymidine or BrdU incorporation (defined as a decrease greater than 2 standard deviations from the mean of untreated control cells). Data from the 29 IC patients and 33 controls indicated a sensitivity of 76% and specificity of 94% for ³H-thymidine incorporation, and data from 16 IC patients and 16 controls indicated a sensitivity of 75% and a specificity of 88% for BrdU incorporation (Table 1). The positive predictive value and negative predictive value were 92% and 82% for ³H-thymidine incorporation and 86% and 78% for BrdU incorporation, respectively.

Data from the expanded studies using 54 IC patients and 34 asymptomatic controls indicated a sensitivity of 85% and a specificity of 91% for ³H-thymidine incorporation. The positive predictive value and negative predictive value were 94% and 80% for ³H-thymidine incorporation.

TABLE 1
Inhibition of Bladder Cell Proliferation as a Diagnostic Assay for IC

| | ³H-Thymidine | |
|---|---|---|
| | Incorporation | |
| | Initial Studies/ | |
| | Expanded Studies | BrdU Incorporation |
| Sensitivity | 76%/85% | 75% |
| Specificity | 94%/91% | 88% |
| Positive Predictive Value | 92%/94% | 86% |
| Negative Predictive Value | 82%/80% | 78% |

Demonstration of the antiproliferative effect required prior serum starvation of the HBE cells and more than 24 hours of exposure to IC urine. These requirements are consistent with an effect on the process of cell proliferation rather than a directly toxic effect. The lack of a difference in trypan blue exclusion between cells exposed to IC or control urine, or evidence for apoptotic DNA breakdown in cells exposed to IC urine, support this hypothesis (data not shown).

The effect of serial dilution of urine on ³H thymidine incorporation was then examined, to determine whether the decreased incorporation in response to IC urine resulted from lack of a urine growth factor(s) or presence of an inhibitory factor(s). The greatest inhibition of ³H-thymidine incorporation occurred in response to the highest concentration of urine from IC patients; serial dilution of the inhibitory effect suggested the presence of an antiproliferative factor (APF) in IC urine.

Additional evidence that the urine of IC patients contains a factor that actively inhibits bladder epithelial cell proliferation was provided by recent experiments that demonstrated net inhibition of ³H-thymidine incorporation in response to the addition of equal volumes of IC urine and control urine to the cell medium. More specifically, HBE cells were cultured in the presence of 1) the less than 10 kD fractionation of urine from either of 2 IC patients, 2) the less than 10 kD fraction of urine from their age-, race-, and sex-matched controls, 3) a combination of equal parts of less than 10 kD fractions of IC and control urine, or 4) serum-free cell culture medium alone, for 48 hours prior to performance of the cell proliferation assay. Specimens were diluted in serum-free medium such that the final concentration of either IC or control urine was the same in each well that contained a particular specimen. Data are expressed as the mean percent change in ³H-thymidine incorporation for cells cultured with urine specimens as compared to cells cultured in serum-free culture medium alone (FIGS. 1D and 1E).

Inhibition of T24 carcinoma cell proliferation by IC patient urine specimens

T24 bladder carcinoma cells (ATCC #4-HTB) (Rockville, Md.) were grown in McCoy's medium containing 10% fetal bovine serum (FBS), 1% antibiotic/antimycotic solution, and 1% glutamine (Sigma, St. Louis, Mo.). These cells were seeded onto 96 well tissue culture plates (Corning Glass Works, Coming, N.Y.) at a density of 5×10³ cells/well, and incubated overnight. The pH of IC patient or control urine specimens was adjusted to 7.0 by the addition of 10 N NaOH or 1 N HCl, and the osmolality was adjusted to 300 mOsm by the addition of 1 M NaCl or distilled H₂O. Varying dilutions of urine specimens in standard culture medium were added to the cells, which were then incubated further at 37° C. for 48 hours prior to performance of the ³H thymidine incorporation assay.

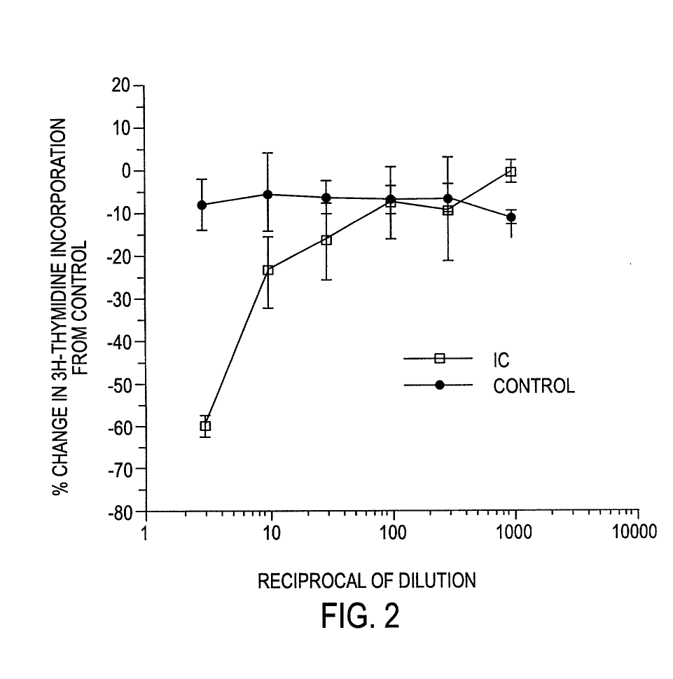

IC urine specimens inhibited proliferation of T24 cells (which were derived from a malignant human tumor and do not have a finite life span in vitro) (FIG. 2). Control urine specimens did not inhibit the proliferation of T24 cells. These data are consistent with APF inhibition of the proliferation of both normal and immortalized cells, and with the use of APF to control tumor cell proliferation.

7.1.4 Characterization of APF

Stability in a Freeze-Thaw Cycle. Studies to determine the stability of the APF in IC urine indicated this factor was fairly stable in a freeze-thaw cycle, with only 18.5+/−8.2% loss of activity. Heating of the IC urine specimens resulted in unchanged antiproliferative activity (% change in ³H-thymidine incorporation of cells incubated with IC urine compared to cells incubated with medium alone =−62.0 +/−6.8 for unheated specimens, to −60.7 +/−14.0 for specimens heated to −70° C. for 2 hours).

Susceptibility to Trypsin Degradation. To determine whether the APF is proteinaceous, its susceptibility to proteases was also examined. Trypsinization of seven IC urine specimens effectively removed most of the antiproliferative activity (D=loss of 87.5+/−26.7% of antiproliferative activity compared to untrypsinized control exposed to the same incubation conditions).

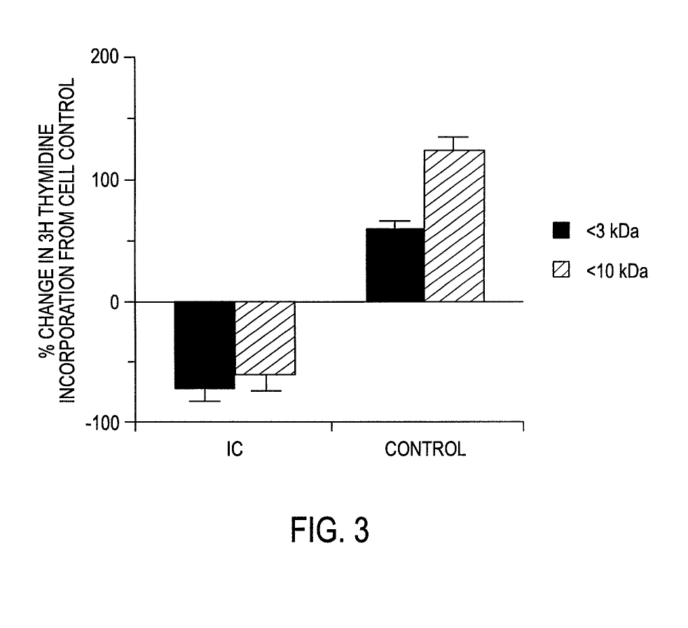

Dialysis and Size Fractionation. The approximate size of the putative antiproliferative protein was initially determined by dialysis. Dialysis of substances less than 10,000 daltons resulted in effective removal of the APF, while dialysis of substances less than 1000 daltons retained the factor. These findings were confirmed by fractionation of the inhibitory IC specimens using “CENTRIPREP” filters; IC urine fractions of substances <10 kDa inhibited bladder epithelial cell proliferation to the same degree as whole urine, while the same fractions from controls were stimulatory. These data are consistent with a conclusion that the antiproliferative effect of IC patient urine is due to the presence of a 1-10 kDa relatively heat stable protein(s). Other experiments using the fraction filtered through a 3000 dalton cut-off “CENTRIPREP” filter have indicated that the molecular weight range for the APF is actually 1-3 kDa (FIG. 3).

Ion-exchange Chromatography. The <10,000 dalton fraction of 500 ml of urine from an IC patient was loaded onto a “MONO Q” sepharose preparative column and components eluted with 1 M NaCl. Each fraction was then tested for its ability to inhibit ³H-thymidine incorporation into normal human bladder cells. Data in FIG. 5 are expressed as the mean % inhibition of ³H-thymidine incorporation in cells incubated with IC patient urine specimens compared to cells incubated with serum-free cell culture medium alone. Each data point is the mean of three samples; bars indicate standard error of the mean. The line indicates the osmolarity generated by the NaCl gradient.

Using this method of peptide purification several fractions (contained within one broad peak) were eluted which demonstrated antiproliferative activity, indicating that ion-exchange chromatography is a useful preliminary purification step for APF.

Hvdrophobic Interaction Chromatography. The <10,000 dalton fraction of urine from an IC patient was loaded onto a phenyl sepharose 6 fast flow (high sub) column in 1 M ammonium sulfate buffer, and components were eluted using 50 mM sodium phosphate buffer (pH 7.0). Each fraction was then tested for its ability to inhibit ³H-thymidine incorporation into normal human bladder cells. Data in FIG. 6 are expressed as % change in cpm of cells incubated with IC patient urine specimens compared to cells incubated with serum-free cell culture medium alone. Each data point is the mean of three samples; bars indicate standard error of the mean.

Using this purification method, a single fraction was obtained with significant inhibitory activity, indicating that this method of hydrophobic interaction chromatography subsequent to ion-exchange chromatography is useful for further purification of the APF.

High Performance Liquid Chromatography. Preliminary attempts to purify APF from the <10 kD urine fraction of five IC patients by HPLC indicated the presence of a single peak fraction that inhibited ³H thymidine incorporation into HBE cells (data shown for one patient, FIG. 4). No inhibitory fraction was identified in the <10 kD fraction from five age-, race-, and sex-matched controls.

Following ion-exchange chromatography and hydrophobic interaction chromatography, HPLC was used as a final step in the purification scheme of APF. This scheme yielded several peaks at absorbance 215/280 nm. FIG. 7A shows the HPLC acetonitrile elution profiles (215/280 nm absorbance) of IC patient urine with active antiproliferative fraction, indicated by the arrow. The single fraction with antiproliferative activity was reapplied to the HPLC column, and the elution profile which indicates purification to homogeneity is indicated by FIG. 7B.

The same purification scheme, employing ion-exchange chromatography, hydrophobic interaction chromatography, and HPLC, has been applied to the less than 10 kDa fraction of urine from control patients. Mock APF fractions from the HPLC acetonitrile elution of control patient specimens failed to show any antiproliferative activity by the inhibition of ³H-thymidine incorporation assay.

MALDI-TOF mass spectrometric analysis was performed on a PerSeptive Biosystems (Framingham, Mass.) Voyager. Mass calibration was done using as standards angiotensin I, ACTH (clip 1-17), ACTH (clip 18-39), ACTH (clip 7-38) and bovine insulin (PE Biosystems, Foster City, Calif.). A cyano-4-hydroxy-cinnaminic acid (Aldrich Chemical Co., Milwaukee, Wis.) at 10 mg/ml in 30% acetonitrile / 0.3% trifluoroacetic acid was used as the matrix. Using that methodology, the APF has a molecular mass of slightly less than 2.5 kDa.

Amino Acid Analysis. APF has 2 asparagines/aspartic acids, 1 threonine, 2 serines, 4 glutamines/glutamic acid, 1 proline, 4 glycines, 2 alanines, 1 valine, 1 isoleucine, 2 leucines, 1 tyrosine, 1 phenylalanine, 1 lysine, 1 arginine, and at least one cysteine. One of the lysine residues may be modified.

7.1.5 Discussion

Example I describes the regimen of experimentation leading to the discovery and initial characterization of APF. APF is an antiproliferative factor in the urine of IC patients that inhibits the proliferation of primary normal human bladder epithelial cells in vitro as determined by ³H-thymidine incorporation.⁵⁵ Urine from 50 of 58 (86%) women with IC significantly inhibited human bladder epithelial cell proliferation as compared to urine from 3 of 36 (8%) asymptomatic control women, 7 of 58 (12%) women with bacterial cystitis, and 0 of 12 (0%) women with vulvovaginitis (p<.001 for the comparison of mean percent change in 3H-thymidine incorporation with IC urine vs. urine from each of the control groups). Additionally, studies using catheterized urine specimens from the bladder and renal pelvis of IC patients are consistent with a conclusion that the APF is made and/or activated in the distal ureter or urinary bladder.⁵⁶

### 7.2 EXAMPLE II

Because of the role growth factors play in epithelial cell proliferation, the inventors also measured levels of urine growth factors known to be important for maintenance of a healthy epithelium and found complex changes in the levels of specific factors in IC patients.⁵⁷ Urine heparin-binding epidermal growth factor-like growth factor (HB-EGF) levels were specifically and significantly decreased in IC patients as compared to asymptomatic controls or patients with bacterial cystitis, whether expressed as concentration (amount per volume of urine) or the amount relative to urine creatinine in each specimen. In contrast, urine epidermal growth factor (EGF), insulin-like growth factor 1 (IGF1), and insulin-like growth factor binding protein 3 (IGFBP3) levels were all elevated in IC patients compared to asymptomatic controls or patients with bacterial cystitis (when expressed per milligram of urine creatinine).

Based on these findings, the inventors hypothesized that IC results from inhibition of normal bladder epithelial regeneration by the APF, which may be mediated by regulation of growth factor production.⁵⁸ To learn more about the APF and its role in IC, the inventors have characterized the APF further, determining a specific source and molecular mass (described in Example I). The inventors also demonstrated that purified APF specifically down-regulates the production of HB-EGF by bladder epithelial cells in vitro and that recombinant HB-EGF can inhibit the antiproliferative effects of the APF on bladder epithelial cells in a dose-dependent manner. These findings provide additional evidence for a role for the APF in the etiology of IC, and are consistent with a mechanism whereby APF inhibits bladder epithelial cell proliferation.

7.2.1 Methods and Materials

Subjects. All IC patients had previously undergone diagnostic cystoscopy and fulfilled the NIDDK diagnostic criteria for IC⁵⁹. Controls included asymptomatic age- (+5 years), race- and sex-matched individuals as well as patients who were scheduled to undergo cystoscopy for other urological diseases. All participants were at least 18 years old and were enrolled in accordance with guidelines of the Institutional Review Board of the University of Maryland School of Medicine.

Urine Specimens. Urine was collected by the clean catch method in which each IC patient or control wiped the labial area with 10% povidone iodine solution and then collected a midstream urine into a sterile container, as previously described⁶⁰. Specimens were initially kept at 4° C., transported to the laboratory where cellular debris was removed by low speed centrifugation at 4° C., aliquoted under sterile conditions and stored at −80° C. until used.

Serum Specimens. Blood was collected from 8 IC patients and 10 age- and sex-matched controls and allowed to clot at room temperature for 30 minutes. Serum was then removed from each specimen and stored at −20° C. until used.

Cell Culture. Cystoscopy was performed under general anesthesia employing nonbacteriostatic normal saline as a bladder irrigant. Rigid cold cup biopsy forceps (Olympus Corp., Lake Success, N.Y.) were used to acquire 4 mm² pieces of transitional epithelium with submucosa from both IC patients and one control for the growth of primary bladder epithelial cells; in addition, epithelial cells were also grown from bladder tissue obtained at autopsy from two patients who had no history of bladder disorder. Tissue specimens were transported from the operating room or pathology department in sealed sterile containers containing sterile phosphate-buffered saline at room temperature, then removed and placed into Eagle's minimal essential medium (MEM) for growth of bladder epithelial cell explants [characterized by binding of AE-1/AE-3 pancytokeratin antibodies (Signet, Dedham, Mass.), as previously described⁶¹]. The epithelial cells were grown in MEM containing 10% heat inactivated FBS, 1% antibiotic/antimycotic solution, 1% glutamine, and 1.0 U/ml insulin (all from Sigma), at 37° C. in a 5% CO₂ atmosphere.

³H-Thymidine Incorporation. Primary normal adult human bladder epithelial cells (HBE) were grown from biopsies obtained at autopsy from patients who had no history of bladder disorder. HBE cells were plated at a density of 1×10⁴ cells/well onto 96 well tissue culture plates and incubated at 37° C. overnight (resulting in approximately 60% confluence the following day). The medium was then changed to MEM containing only 1% glutamine and 1% antibiotic/ antimycotic solution, and the cells were incubated at 37° C. overnight. On the third day urine specimens from IC patients or controls were corrected to pH 7.2 and 300 mOsm, filtered through a 0.2 mm pore filter (Gelman Sciences, Ann Arbor, Mich.), diluted 1:2 in MEM (“serum-free MEM” containing only glutamine and antibiotics/antimycotics) and applied to the cells; cell controls received serum-free MEM alone. Specimens from chromatography fractions (see below) were applied directly to the cells (5 1/200 1 serum-free medium) to test for antiproliferative activity. Following 48 hours of incubation at 37° C., the cells were pulsed with 1 μCi/well ³H-thymidine (NEN DuPont, Wilmington, DE) and incubated for another 4 hours at 37° C. Cells were then trypsinized, and insoluble cell contents harvested and methanol-fixed onto glass fiber filter paper, as previously described⁶²; the amount of radioactivity incorporated was determined as counts per minute using a Beckman LS 3801 scintillation counter. A significant inhibition of ³H-thymidine incorporation was defined as a mean decrease in counts per minute of greater than 2 standard deviations from the mean of control cells for each plate.

ELISAs. Bladder epithelial cells explanted from IC or control bladder tissue specimens were seeded at 5×10⁴ cells per well of a 24 well tissue culture plate (Corning) or at 5×10⁶ cells per 75 mm 2 tissue culture flask (Becton Dickinson, Franklin Lakes, N.J.) and grown to approximately 80% confluence. Cell culture medium was then changed to MEM containing only 1% glutamine and 1% antibiotic/antimycotic solution, and the cells were incubated at 37° C. in a 5% CO₂ atmosphere overnight. The next day IC cell supernatant, low molecular weight fraction of urine, or purified APF was applied, and the cells were incubated for an additional 48 hours. Supernatant was removed from these cultures and frozen at −80° C. until determination of growth factor levels by ELISA.

HB-EGF - Cell supernatants or patient serum samples (200 1) were applied to each well of a 96 well Immulon II plate (Dynatech Laboratories, Chantilly, Va.) at 40° C. overnight. Following 5 washes with phosphate buffer the plates were blocked with 5% fetal bovine serum/1 mM EDTA/0.05% Tween 20 in PBS. Anti-HB-EGF antibody (1 mg/ml) (R & D Systems, Minneapolis, Minn.) was added and the plates were incubated for 2 hours at 37° C. Following an additional 5 washes, biotinylated anti-goat IgG/avidin D horseradish peroxidase was added and plates were incubated for 1.5 hours at room temperature, washed, and developed with ABTS [2,2′-Azino-bis-(3-ethylbenzothiazoline-6-sulfonic)] substrate; absorbance was read at 405 nm.

EGF - Cell supernatants or serum samples were diluted 1:200-1:300 in RD5E diluent and pipetted into wells precoated with monoclonal anti-EGF antibody, according to the manufacturer's instructions (R & D Systems, Minneapolis, Minn.). Following incubation at room temperature for 4 hours, plates were washed with phosphate buffered saline (PBS) and incubated further with HRP-linked polyclonal anti-EGF, washed, and developed with tetramethylbenzidine (TMB) substrate; development was stopped with 0.2 M H₂SO₄, and absorbance read at 450 nm.

IGF1 - Cell supernatants were concentrated 30-fold by lyophilization and reconstitution in ethanolic HCl in accordance with published recommendations⁶³. After 30 minutes incubation at room temperature, samples were centrifuged at 10,000 rpm for 3 minutes to remove insoluble material, and supernatant neutralized to pH 7 with neutralization buffer. Neutralized, extracted samples were added to wells along with anti-IGF HRP-conjugate (Diagnostic Systems Laboratories, Webster, Tex.), and plates were incubated for 2 hours at room temperature. Following washes, plates were developed with TMB chromagen solution; development was stopped with 0.2 M H₂SO₄, and absorbance read at 450 nm.

IGFBP3 - Cell supernatants were added to wells precoated with polyclonal anti-IGFBP3 (Diagnostic Systems Laboratories, Webster, TX), then incubated at room temperature for 2 hours. Following washes, another polyclonal, HRP-labeled anti-IGFBP3 antibody was added to the wells, and the plates were further incubated, washed, and developed with TMB substrate; development was stopped with 0.2 M H₂SO₄ and absorbance read at 450 nm.

Linear absorbance vs. concentration curves were prepared from results with known standard concentrations of EGF, HB-EGF, IGF1 or IGFBP3, and sample concentrations were determined by plotting absorbance values.

³⁵S -Methionine Incorporation. Primary normal human bladder epithelial cells were plated at a density of 5×10⁴ cells/well onto 24 well culture plates (Corning) and incubated at 37° C. in a 5% CO₂ atmosphere overnight. The next day the medium was changed to serum-free MEM containing only 1% glutamine and 1% antibiotic/antimycotic solution and the cells were again incubated overnight. On the third day, purified APF was added to the cell medium along with 2.5 μCi ³⁵S methionine, and the cells were incubated for an additional 48 hours. The cell medium was then removed and frozen at −80° C. for subsequent determination of growth factor concentration by ELISA. The cells were trypsinized, lysed with deionized/distilled water, and insoluble cell contents harvested and fixed with 10% trichloroacetic acid onto glass fiber filter paper using a cell harvester (Hoefer Scientific Instruments, San Francisco, Calif.); the amount of radioactivity incorporated was determined as counts per minute using a Beckman LS 3801 scintillation counter.

Molecular Weight Fractionation. All urine samples underwent low speed centrifugation at 40° C. for 5 minutes to remove cellular debris. The acellular urine was then filtered through a Centriplus 10 filter (<10,000) (Amicon, Inc.) by centrifugation at 1700 x g for approximately 3 hours (until only 5% of the specimen remained in the upper chamber). The filtered urine fractions were then stored at −80° C. until used.

Ion Exchange Chromatography. Low molecular weight fractions of urine or whole bladder epithelial cell supernatants were diluted 1:1 with 20 mM sodium phosphate buffer (pH 7.0) and applied to a Q-sepharose (Sigma, St. Louis, Mo.) column at 40° C. Proteins were then eluted using 1 mM sodium chloride/20 mM sodium phosphate buffer (pH 7.0) gradient (0-100% gradient over 120 minutes).

Hydrophobic Interaction Chromatography. Fractions from the ion exchange chromatographic purification determined to have antiproliferative activity were pooled, diluted 1:1 with 2M ammonium sulfate buffer (pH 7.0), filtered through a 0.2 mm filter, and applied to a phenyl-sepharose column (Amersham Pharmacia Biotech, Inc., Piscataway, N.J.). Components were eluted with 50 mM sodium phosphate/1 M ammonium sulfate buffer (pH 7.0) gradient (0-100% gradient over 120 minutes).

Reversed-phase High Performance Liquid Chromatography (HPLC). Fractions from the hydrophobic interaction chromatography purification determined to have antiproliferative activity were applied to a Vydac 201HS C18 column (The Separations Group, Hesperia, Calif.) and peptides eluted with 100% acetonitrile (5-75% gradient over 70 minutes). Fractions were immediately aliquotted, and either applied to the cells for the cell proliferation assay or freeze-dried and stored under nitrogen at −80° C.

Protein Quantification. The amount of protein present in each sample was determined using the Lowry assay⁶⁴.

Mass Spectrometry. MALDI-TOF mass spectrometric analysis was performed on a PerSeptive Biosystems (Framingham, Mass.) Voyager. Mass calibration was done using as standards angiotensin I,

ACTH (clip 1-17), ACTH (clip 18-39), ACTH (clip 7-38) and bovine insulin (PE Biosystems, Foster City, Calif.). A cyano-4-hydroxy-cinnaminic acid (Aldrich Chemical Co., Milwaukee, Wiss.) at 10 mg/ml in 30% acetonitrile / 0.3% trifluoroacetic acid was used as the matrix.

Statistical Analysis. Comparison of mean change in ³H-thymidine incorporation caused by urine specimens from IC patients vs. controls was performed using a two-tailed analysis of covariance. Comparison of growth factor levels between IC and control groups was performed using Student's t test.

7.2.2 Production of APF by Bladder Epithelial Cells from IC Patients.

The inventors observed that catheterized urine specimens collected from the bladder of 20 female IC patients had antiproliferative activity significantly more often than specimens collected from the renal pelvis of the same patients during the same procedure,⁶⁵ consistent with a conclusion that APF is produced within the lower urinary tract. Based on this observation, the inventors proceeded to determine whether primary bladder epithelial cells from IC patients produce APF in vitro.

Spent culture medium of epithelial cells grown from the bladder biopsies from 6 of 6 IC patients (3 female and 3 male) contained an antiproliferative factor that inhibited the proliferation of normal bladder epithelial cells as compared to cell medium alone (mean % change in ³H-thymidine incorporation=−85.0 +2.0), while spent culture medium of cells grown from 3 of 3 control patients did not inhibit the growth of the same normal epithelial cells (+11.3+7.2, p<.01).

7.2.3 APF Purification

APF was purified to homogeneity (FIG. 7B) from each of 8 IC patient urine specimens as well as from the supernatants of bladder epithelial cell explants from 5 additional IC patients. The purification scheme included:

sequential size exclusion filtration;

ion exchange chromatography;

hydrophobic interaction chromatography; and

reversed-phase HPLC.

Fractions with antiproliferative activity from each of the 13 specimens contained a substance with absorbance at 215 and 280 nm which eluted at the same point in each elution gradient. Based on biological activity and amount of protein, this purification scheme yielded an approximately 7000 fold increase in specific activity from whole urine specimens and an approximately 2000 fold increase in specific activity from cell supernatants (Table 2).

In comparison, urine specimens from 3 asymptomatic persons who were age-, race- and sex-matched controls for 3 of the IC patients (including one specimen that contained antiproliferative activity by the cell proliferation assay) were also subjected to the same purification scheme using fractions from the same part of each elution gradient; final HPLC purification of these specimens yielded no evidence for protein or antiproliferative activity.

TABLE 2
Specific Activity of APF at Various Stages of Purification.

| | IC50 | | Fold Purification |
|---|---|---|---|
| IC Patient Urine | | | |
| Whole Urine | 8.4 | μg (undiluted) | — |
| <10 kDa Fraction | 7.8 | μg (undiluted) | 1.08 |
| Mono-Q Sepharose Fractions | 4.6 | μg | 1.82 |
| Phenyl-Sepharose Fractions | 0.4 | μg | 21 |
| C18-HPLC Fractions | 1.2 | ng | 6,666.67 |
| IC Bladder Cell Supernatant | | | |
| Whole Supernatant | 7.5 | μg | |
| Mono-Q Sepharose Fractions | 0.6 | μg | 12.50 |
| Phenyl-Sepharose Fractions | 0.05 | μg | 150.00 |
| C18-HPLC Fractions | 4.4 | ng | 1,704.00 |

7.2.4 Characterization of APF

Mass Spectrometry. MALDI-TOF mass spectrometry revealed a single peak with molecular mass ranging from 2485.2 to 2592.23 in five different samples (1 urine specimen and 4 supernatant specimens) from three different patients (FIG. 8).

Amino Acid Analysis. In addition to determining trypsin-sensitivity, heat stability, molecular mass, and a source of production for the APF, the inventors have also obtained important information regarding the structure of APF. Purity of the specimen was first confirmed by mass spectrometric analysis. Amino acid analysis indicated the presence of aspartate/asparagine, glutamate/glutamine, glysine, valine, alanine, serine and leucine residues.

N-terminal amino acid sequencing was blocked, however, requiring internal sequencing of the peptide. Mass spectrometric analysis using both LC/MS and LC/MS/MS technology was used to analyze fragments of the APF generated by ion bombardment and trypsinization. This analysis confirmed the definite presence of amino acids. A 4-amino acid stretch was obtained: gly-gly-ala-hydroxylysine. The amino acids present are 2 asparagines/aspartic acids, 1 threonine, 2 serines, 4 glutamines/glutamic acid, 1 proline, 4 glycines, 2 alanines, 1 valine, 1 isoleucine, 2 leucines, 1 tyrosine, 1 phenylalanine, 1 lysine, 1 arginine, and at least one cysteine.

NMR Analysis. The presence of peptide moieties was confirmed by NMR analysis. HPLC-purified APF was lyophilized, resuspended in D₂O, and analyzed on a Varian VXR-500s/unity 500 MHz NMR spectrometer. This sample was run using a preset pulse sequence (with irradiated water peak) and a Nalorac MIDG-3mm probe.

The location of specific peaks indicate the presence of various molecules to which hydrogen is attached. The spectrum obtained for APF indicates the definite presence of amino groups (indicated by “A” peak at 6.6) and an aromatic (probably tyrosine) structure (indicated by three peaks in “B” between 7 and 8). In addition, several other peaks indicate the presence of saturated carbon-hydrogen groups (indicated by peaks in “C” between 3 and 4). The major peak at 4.8 is generated by the deuterium in the solvent.

Presence of these same specific groups has now been confirmed by NMR using the specimen from another IC patient. Further analysis of the complete APF structure can be obtained using additional one-dimensional analysis of the major peaks as well as two-dimensional NMR, using routine procedures known to those of skill in the art.

7.2.5 Inhibition of Bladder Epithelial Cell HB-EGF Production by HPLC-Purified APF

Serum-starved bladder epithelial cells from IC patients were determined to produce significantly lower levels of HB-EGF and higher levels of EGF and IGF1 into the culture medium than bladder cells from controls (p<.05 for a comparison of each growth factor concentration produced by cells from both IC patients compared to the concentration produced by cells from the normal control).

Based on these observations, the inventors proceeded to determine whether APF itself could cause changes in bladder epithelial cell growth factor production. Primary normal human bladder epithelial cells exposed to HPLC-purified APF produced approximately 80-90% less HB-EGF and approximately 80-100% more EGF, IGF1, and IGFBP3 into the cell medium than cells incubated with serum-free medium alone or mock APF purified from the urine of controls (p<.01 for differences in production of each growth factor in response to APF vs. mock control). Because the APF also caused approximately 20-25% inhibition of generalized protein synthesis as determined by ³⁵S-methionine incorporation, growth factor and binding protein levels were normalized to incorporation of radioactivity.

7.2.6 Blocking of APF Activity by rHB-EGF

To determine whether the antiproliferative effect of the APF on bladder epithelial cells might be due to a decrease in HB-EGF production, primary normal bladder epithelial cells were simultaneously incubated with varying levels of recombinant HB-EGF, EGF, or IGF1 and low molecular weight urine fractions from IC patients. IC urine antiproliferative activity was inhibited by HB-EGF in a dose-dependent manner, suggesting that the APF activity is mediated by downregulation of HB-EGF production. This effect was evident using a concentration of HB-EGF (10 ng/ml) in the range previously determined for normal controls (2-22 ng/ml, mean 6.33 ng/ml) 12 as well as higher concentrations (30 and 100 ng/ml). In fact, at the higher concentrations of rhHB-EGF the proliferation of cells exposed to urine from IC patients was stimulated more than that of cells exposed to control urines. In comparison, even the two highest concentrations of recombinant EGF or IGF1 tested [corresponding to approximately 12 and 25 fold greater concentrations than previously determined for normal controls 12] were insufficient to completely inhibit APF activity.

Confirmation that rhHB-EGF can inhibit APF activity was achieved by adding HPLC-purified APF to the medium of cells derived from both normal and IC patient bladder biopsies. Using the lowest concentration of purified APF (10 ng) predetermined to inhibit normal bladder epithelial cell ³H-thymidine incorporation by more than 50%, antiproliferative activity was inhibited for 2 of 3 APF specimens by 30 ng/ml of HB-EGF in both normal and IC patient cells.

7.2.7 Serum HB-EGF and EGF Levels in IC Patients

As an indirect means of determining whether the APF and its effects on HB-EGF production are confined to the urinary epithelium in IC patients or whether IC may be a urinary tract manifestation of a systemic process, the inventors determined the quantity of immunoreactive HB-EGF in serum from 8 IC patients and 10 normal controls. The concentration of HB-EGF was strikingly lower in IC patient specimens (2.3 +0.1 ng/ml) as compared to asymptomatic controls (9.1+0.9 ng/ml) (p<.001). In comparison, serum EGF levels were significantly higher in IC patients (23.3+3.4 ng/ml) than controls (11.8+1.0) (p<.001).

7.3 Discussion

Interstitial cystitis is a disabling disorder of both women and men that is associated with a distinct set of clinical symptoms. Although numerous theories have been proposed to explain its pathogenesis, no specific etiology has been found.

The inventive work set forth in Example I (Section 7.1) show that urine from women with IC inhibits human bladder epithelial cell DNA synthesis significantly more often than urine from asymptomatic control women, women with acute bacterial cystitis or women with vulvovaginitis⁶⁶. These findings are consistent with a conclusion that the urine of affected patients lacks one or more growth factors and/or contains an inhibitor of cell growth. The inventive work described in Example II (Section 7.2) indicate that the decrease in bladder epithelial cell DNA synthesis caused by IC urine results from alterations in epithelial growth factor levels mediated by a specific APF, which has now been purified to homogeneity and found to have a molecular mass of approximately 1.7 or 2.5 kDa, depending on the technique and apparatus used to determine the molecular mass. APF has been purified from 8 IC patient urine specimens as well as from the supernatant of bladder epithelial cell explants from 5 IC patients using the same purification process, and 5 specimens examined by mass spectrometry were found to have essentially the same molecular mass; these findings make it likely that the same factor is made by bladder epithelial cells from various IC patients. Even the measurement of APF activity in urine provides a very sensitive and specific test for IC⁶⁷. These findings are consistent with a conclusion that IC is either a single disease caused by APF production, or it is more than one disease with a final common pathway involving APF production.

The work described herein and the known thinning or denudation of the bladder epithelium common in IC patients⁶⁸ are consistent with APF regulation of epithelial cell growth factor in the pathogenesis of IC. Moreover, the markedly lower levels of HB-EGF in the urine of IC patients than controls, but higher levels of EGF, IGF1 and IGFBP3 ⁶⁹, are consistent with APF regulation of these and possibly other epithelial growth factors. Purified APF is a negative autocrine growth factor which downregulates HB-EGF production while stimulating the production of EGF, IGF1 and IGFBP3.

APF production by IC patient bladder epithelial cells and systemic differences in growth factor levels between IC patients and controls are consistent with an endogenous or genetic pathogenesis for IC. Although few studies have explored a possible genetic origin for this disease, a familial clustering has been reported with 6-7% of IC patients having family members with the same disease, making IC roughly 100 times more common in relatives than the general population.⁷⁰

The fact that purified APF alone is capable of affecting bladder epithelial cell production of at least 3 epithelial cell growth factors (HB-EGF, EGF, and IGF1) and 1 growth factor binding protein (IGFBP3) is consistent with a conclusion that the analogous differences in urine⁷¹ and serum levels of these proteins between IC patients and controls is caused either directly or indirectly by the APF. Various components of the EGF and IGF systems undergo a similar coordinated regulation during tissue repair in other organs, but in the opposite direction. For example, following acute injury to the rat kidney there is an upregulation of HB-EGF and transforming growth factor-alpha gene expression and protein synthesis⁷² while EGF, IGF1 and IGFBP3 mRNA are coordinately reduced⁷³. A similar pattern is seen in experimental cerebral injury, in which HB-EGF mRNA is upregulated while EGF mRNA is undetectable⁷⁴. Furthermore, EGF can stimulate the synthesis of IGF1⁷⁵, and IGFBP3 synthesis can also be regulated by other growth factors such as transforming growth factor beta 1 (TGF-,β1)⁷⁶. These findings are consistent with mechanisms involving specific roles for all four of these growth regulating peptides in a variety of tissues and that their synthesis is often controlled in a coordinate manner. APF is the first substance reported to regulate HB-EGF, EGF, IGF1 and IGFBP3 production in a direction opposite to that seen in tissue repair. In addition to the clinical applications for APF and its functional equivalents described herein, this discovery therefore provides an interesting new opportunity to gain additional fundamental insights into the control of cell growth and tissue repair.

8. References

Throughout this specification various patent and nonpatent references have been cited. The entire disclosure of each of these references is incorporated herein by reference, specifically including without limitation the entire disclosure of the following listed references:

¹ Ratner, et al. (1994) Urol. Clin. North Am. 21:1-5; Hanno et al. Epidemiology of interstitial cystitis: 2. In: (1990) Interstitial Cystitis, London: Springer-Verlag.

² Oravisto, K. J. (1975) Ann. Chir. Gynaecol. Fenn. 64: 75.

³ Johansson and Fall (1990) J. Urol 143:1118; Smith and Dehner (1972) Arch. Pathol 93:76.

⁴ Oravisto, ibid; Smith, ibid.

⁵Fowler et al. (1988) J. Urol. 140:1385.

⁶ Hanno, ibid.

⁷ For review, see Robbins and Angell, 1976, Basic Pathology, 2d Ed., W. B. Saunders Co., Philadelphia, pp. 68-122.

⁸ Lemer, Advances in Metastatic Bladder Cancer, www.medscape.com/medscape/cno/2000/AUA/Story.cfm?story_id=1415.

⁹ Lerner, Advances in Metastatic Bladder Cancer, www.medscape.com/medscape/cno/2000/AUA/Story.cfm?story_id=1415.

¹⁰ Lerner, Advances in Metastatic Bladder Cancer, www.medscape.com/medscape/cno/2000/AUA/Story.cfm?story id=1415.

¹¹ Kohler et al. Nature 256:495 (1975); Eur. J. Immunol. 6:511 (1976); Euro J Immunol. 6:292 (1976).

¹² See, e.g., Hudson & May, 1986, Practical Immunology, Blackwell Scientific Publications, Oxford, United Kingdom.

¹³ See, e.g., O'Connor et al., 1994, Endocrine Reviews 15:650-683; Krichevsky et al, 1991, Endocrinology 128:1255-1264; and Krichevsky et al., 1988, Endorcrinology 123:584-593.

¹⁴ Krichevsky et al., 1988, Endocrinology 123:584-593.

¹⁵ See, e.g., Sambrook et al., 1989, Molecular Cloning, A Laboratory Manual, Cold Spring Harbor Laboratory, 2d Ed., Cold Spring Harbor, New York, Glover, D. M. (ed.), 1985, DNA Cloning: A Practical Approach, MRL Press, Ltd., Oxford, U.K., Vol. I, II.

¹⁶ Molecular Cloning:A Laboratory Manual, Cold Spring Harbor Laboratory Press, Cold Spring Harbor, N.Y. (1989).

¹⁷ Guide to Molecular Cloning Techniques, Academic Press, Inc., San Diego, Calif. (1987).

¹⁸ Recombinant DNA Laboratory Manual, Academic Press, Inc., San Diego, Calif (1999); Baumberg, ed. Prokaryotic Gene Expression (Frontiers in Molecular Biology), Oxford Univ Press (1999).

¹⁹ Bernoist and Chambon, 1981, Nature 290:304-310.

²⁰ Yamamoto et al., 1980, Cell 22:787-797.

²¹ Wagner et al.,1981, Proc. Natl. Acad. Sci. USA 78:1441-1445.

²² Brinster et al., 1982, Nature 296:39-42.

²³ Swift et al.,1984, Cell 38:639-646; Ornitz et al., 1986, Cold Spring Harbor Symp. Quant. Biol, 50:399-409; MacDonald, 1987, Hepatology 7:425-515.

²⁴ Hanahan, 1985, Nature 315:115-122.

²⁵ Grosschedl et al., 1984, Cell 38:647-658; Adames et al., 1985, Nature 318:533-538; Alexander et al., 1987, MoL Cell. Biol. 7:1436-1444.

²⁶ Leder et al., 1986, Cell 45:485-495.

²⁷ Pinkert et al., 1987, Genes and DeveL 1:268-276.

²⁸ Krumlauf et al., 1985, Mol. Cell. Biol. 5:1639-1648; Hammer et al., 1987, Science 235:53-58.

²⁹ Kelsey et al., 1987, Genes and Devel. 1:161-171.

³⁰ Mogram et al., 1985, Nature 315:338-340; Kollias et al., 1986, Cell 46, 89-94.

³¹ Readhead et al., 1987, Cell 48:703-712.

³² Sani, 1985, Nature 314:283-286.

³³ Mason et al., 1986, Science 234:1372-1378.

^{34 1975,} Nature 256:495-497.

³⁵ Kozbor et al., 1983, Immunology Today 4:72.

³⁶ Cole et al., 1985, in Monoclonal Antibodies and Cancer Therapy, Alan R. Liss, Inc., pp. 77-96.

³⁷ See, e.g., Erlich et al., 1985, Am. J. Reprod Immunol. Microbiol. 8:48.

³⁸ Kennedy et al., Clin. Chim. Acta 70:1-31 (1976).

³⁹ Schurs, A. H. W. M., et al. Clin. Chim Acta 81:1-40 (1977).

⁴⁰ Work, T. S., et al. North Holland Publishing Company, N.Y. (1978).

⁴¹ Kirkham and Hunter, Radioimmune Assay Method, E. & S. Livingstone, Edinburgh, 1970, pp 199-206.

⁴² Plante, Advances in the Treatment of Metastatic Bladder Cancer, www.medscape.com/medscape/cno/2000/ASCO/Story.cfm?storyjid=1300.

⁴³ See, e.g, Wu and Wu, 1987, J. Biol. Chem. 262:4429-4432.

⁴⁴ See Langer, Science 249:1527-1533 (1990); Treat et al., in Liposomes in the Therapy ofinfectious Disease and Cancer, Lopez-Berestein and Fidler (eds.), Liss, New York, pp. 353-365 (1989); Lopez-Berestein, ibid., pp. 317-327; see generally ibid.

⁴⁵ See Langer, supra; Sefton, CRC Crit. Ref Biomed. Eng. 14:201 (1987); Buchwald et al., Surgery 88:507 (1980);Saudeketal., N. Engl. J. Med. 321:574(1989).

⁴⁶ See Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61(1983); see also Levy et al., Science 228:190(1985); During et al., Ann. Neurol. 25:351(1989); Howard et al., J. Neurosurg. 71:105 (1989).

⁴⁷ See, e.g., Goodson, in Medical Applications of Controlled Release, supra, vol. 2, pp. 115-138 (1984).

⁴⁸ Science 249:1527-1533 (1990).

⁴⁹ Oike, Y. et al. (1982) J. Biol. Chem. 257: 9751-9758; Liu, C. et al. (1983) Int. J. Pept. Protein Res. 21: 209-215.

⁵⁰ Broach & Thomer, Nature 384:14-16 (1996).

⁵¹ Broach & Thorner, Nature 384:14-16 (1996).

⁵² IC [Division of Kidney, Urologic, and Hematologic Diseases (DKUHK) of the National Institute of Diabetes and Digestive and Kidney Diseases NIDDK)(1989) Am J. Kidney Dis. 13:353]

⁵³[Keay et al. (1995) Urology 45:223]

⁵⁴[Trifillis,et al. (1993) In vitrol Cell Dev. Biol. 29A:908]

⁵⁵ Keay S, Zhang C-O, Hise M, Trifillis AL, Hebel JR, Jacobs SC, Warren JW. Decreased 3H-thymidine incorporation by human bladder epithelial cells following exposure to urine from interstitial cystitis patients. J Urol 156: 2073, 1996; Keay S, Zhang C-O, Hise MK, Hebel J R, Jacobs S C, Gordon D, Whitmore K, Bodison S, Gordon N, Warren J W. A diagnostic in vitro assay for interstitial cystitis. Urology 52: 974, 1998.

⁵⁶ Keay S, Warren J W, Zhang C-O, Tu LM, Gordon D A, Whitmore, K E. Antiproliferative activity is present in bladder but not renal pelvic urine from interstitial cystitis patients. J Urol 162: 1487, 1999.

⁵⁷ Keay et al. Concentrations of Specific Epithelial Growth Factors in the Urine of Interstitial Cystitis Patients and Controls. J Urol 158: 1983, 1997.

⁵⁸ Keay S, Warren J W. A hypothesis for the etiology of interstitial cystitis based upon inhibition of bladder epithelial repair. Med Hypotheses 51: 79, 1998.

⁵⁹ Division of Kidney, Urologic, and Hematologic Diseases (DKUHD) of the National Institute of Diabetes and Digestive and Kidney Diseases (NIDDK). Diagnostic criteria for research studies (interstitial cystitis). Am J Kidney Dis 13: 353, 1999.

⁶⁰ Keay S, Zhang C-O, Hise M, Triffillis A L, Hebel J R, Jacobs S C, Warren J W. Decreased 3H-thymidine incorporation by human bladder epithelial cells following exposure to urine from interstitial cystitis patients. J Urol 156: 2073, 1996; Keay S, Zhang C-O, Hise M K, Hebel J R, Jacobs S C, Gordon D, Whitmore K, Bodison S, Gordon N, Warren J W. A diagnostic in vitro assay for interstitial cystitis. Urology 52: 974, 1998; Keay S, Warren JW, Zhang C-O, Tu L M, Gordon D A, Whitmore, K E. Antiproliferative activity is present in bladder but not renal pelvic urine from interstitial cystitis patients. J Urol 162: 1487, 1999.

⁶¹ Trifillis A L, Cui X, Jacobs S, Warren J W. Culture and characterization of normal epithelium from cystoscopic biopsies of human bladder. In Vitto Cell Dev Biol Anim 29A: 908, 1993.

⁶² Keay S, Zhang C-O, Hise M, Triffillis A L, Hebel J R, Jacobs S C, Warren J W. Decreased 3H-thymidine incorporation by human bladder epithelial cells following exposure to urine from interstitial cystitis patients. J Urol 156: 2073, 1996; Keay S, Zhang C-O, Hise M K, Hebel J R, Jacobs S C, Gordon D, Whitmore K, Bodison S, Gordon N, Warren J W. A diagnostic in vitro assay for interstitial cystitis. Urology 52: 974, 1998; Keay S, Warren J W, Zhang C-O, Tu L M, Gordon D A, Whitmore, K E. Antiproliferative activity is present in bladder but not renal pelvic urine from interstitial cystitis patients. J Urol 162: 1487, 1999.

⁶³ Third International Symposium on Insulin-like Growth Factors. Valid measurements of total IGF concentrations in biological fluids. Endocrinology 136: 816, 1995.

⁶⁴ Lowry O H, Rosebrough N J, Farr A L, Randall R R. Protein measurement with the Folin phenol reagent. J Biol Chem 193: 265, 1951.

⁶⁵ Keay S, Warren JW, Zhang C-O, Tu L M, Gordon D A, Whitmore, K E. Antiproliferative activity is present in bladder but not renal pelvic urine from interstitial cystitis patients. J Urol 162: 1487, 1999.

⁶⁶ Keay S, Zhang C-O, Hise M, Trifillis A L, Hebel J R, Jacobs S C, Warren J W. Decreased 3H-thymidine incorporation by human bladder epithelial cells following exposure to urine from interstitial cystitis patients. J Urol 156: 2073, 1996; Keay S, Zhang C-O, Hise M K, Hebel J R, Jacobs S C, Gordon D, Whitmore K, Bodison S, Gordon N, Warren J W. A diagnostic in vitro assay for interstitial cystitis. Urology 52: 974, 1998.

⁶⁷ Even the measurement of APF activity in urine appears provides a very sensitive and specific test for IC

68 Ruggieri M R, Chelsky M J, Rosen S I, Shickley T J, Hanno P M. Current findings and future research avenues in the study of interstitial cystitis. Urol Clin North Am 21: 163, 1994.

⁶⁹ Keay S, Zhang C-O, Kagen D I, Hise M K, Jacobs S C, Hebel J R, Gordon D, Whitmore K, Bodison S, Warren J W. Concentrations of Specific Epithelial Growth Factors in the Urine of Interstitial Cystitis Patients and Controls. J Urol 158:1983,1997.

⁷⁰ Koziol J A, Clark D C, Gittes R F, Tan E M. The natural history of interstitial cystitis: a survey of 374 patients. J Urol 149: 465, 1993; Simon L J, Landis J R, Erickson D R, Nyberg L M, The ICDB Study Group. The Interstitial Cystitis Data Base study: concepts and preliminary baseline descriptive statistics. Urology 49 (Suppl. 5A): 64, 1997.

⁷¹ Keay S, Zhang C-O, Kagen D I, Hise M K, Jacobs S C, Hebel J R, Gordon D, Whitmore K, Bodison S, Warren J W. Concentrations of Specific Epithelial Growth Factors in the Urine of Interstitial Cystitis Patients and Controls. J Urol 158:1983,1997.

⁷² Homma T, Sakai M, Cheng H F, Yasuda T, Coffey R J Jr, Harris R C. Induction of heparin-binding epidermal growth factor-like growth factor mRNA in rat kidney after acute injury. J Clin Invest 96: 1018, 1995. Sakai M, Zhang M, Homma T, Garrick B, Abraham JA, McKanna JA, Harris RC. Production of heparin binding epidermal growth factor-like growth factor in the early phase of regeneration after acute renal injury. J Clin Invest 99: 2128, 1997.

⁷³ Hise M K, Liu L, Papadimitriou J C, Drachenberg C I, Rohan R M. Transforming growth factor-alpha (TGF-alpha) expression during acute renal failure. J Amer Soc Nephr 7: 1658, 1996. Safirstein R, Zelent A Z, Price P M. Reduced preproEGF mRNA and decreased EGF excretion in ARF. Kidney Int 36: 810, 1989.

⁷⁴ Safirstein R, Zelent A Z, Price PM. Reduced preproEGF mRNA and decreased EGF excretion in ARF. Kidney mInt 36: 810, 1989.

⁷⁵ Hise M K, Liu L, Mantzouris N, Rohan R M. Differential mRNA expression of insulin-like growth factor system during renal injury and hypertrophy. Am J Physiol 269 (Renal Fluid Electrolyte Physiol. 38): F817, 1995.

⁷⁶ Kawahara N, Mishima K, Higashiyama S, Tanaguchi N. The gene for heparin-binding epidermal growth factor-like growth factor is stress-inducible: its role in cerebral ischemia. J Cereb Blood Flow Metab 19: 307, 1999.

⁷⁷ Baserga R. Gene regulation by IGF-1. Mol Reprod Dev 35: 353, 1993.

⁷⁸ Bushman TL, Kuemmerle JF. IGFBP-3 and IGFBP-5 production by human intestinal muscle: reciprocal regulation by endogenous TGF-betal. Am J Physiol 275:G1282, 1998.

Other general references that establish provide background information are as follows:

Curhan G C, Speizer F E, Hunter D J, Curhan S G, Stampfer M J. Epidemiology of interstitial cystitis: a population based study. J Urol 161:549,1999.

Ratliff T L, Klutke C G, McDougall E M. The etiology of interstitial cystitis. Urol Clin North Am 21: 21, 1994.

Marikovsky M, Breuing K, Liu P Y, Eriksson E, Higashiyama S. Farber P, Abraham J, Klagsbrun M. Appearance of heparin-binding EGF-like growth factor in wound fluid as a response to injury. Proc Natl Acad Sci USA 90: 3889, 1993.

Beerli R R, Hynes N E. Epidermal growth factor-related peptides activate distinct subsets of ErbB receptors and differ in their biological activities. J Biol Chem 271: 6071, 1996.

Bromberg J F, Fan Z, Brown C, Mendelsohn J, Darnell J E, Jr. Epidermal growth factor-induced growth inhibition requires Statl activation. Cell Growth Differ 9: 505, 1998.

Leal SM, Huang S S, Huang J S. Interactions of high affinity insulin-like growth factor-binding proteins with the type V transforming growth factor-beta receptor in mink lung epithelial cells. J Biol Chem 274: 6711, 1999.

Jones J I, Clemmons D R. Insulin-like growth factors and their binding proteins: biological actions. Endocrine Rev 16: 3, 1995.

Elenius K, Paul S, Allison G, Sun J, Klagsbrun M. Activation of HER4 by heparin-binding EGF-like growth factor stimulates chemotaxis but not proliferation. EMBO J 16: 1268, 1997.

Wang L M, Kuo A, Alimandi M, Veri M C, Lee C C, Kapoor V, Ellmore N. Chen XH, Pierce JH. ErbB2 expression increases the spectrum and potency of ligand-mediated signal transduction through ErbB4. Proc Natl Acad Sci USA 95: 6809, 1998.

The examples provided herein are for illustrative purposes only and are in no way intended to limit the scope of the present invention. While the invention has been described in detail, and with reference to specific embodiments thereof, it will be apparent to one with ordinary skill in the art that various changes and modifications can be made therein without departing from the spirit and scope thereof.

We, the inventors, claim:

1. An antiproliferative composition, wherein:
(a) the composition:
(i) comprises a purified antiproliferative factor;
(ii) comprises a fraction of a source of the antiproliferative factor;
(iii) comprises a functional equivalent of the antiproliferative factor;
(b) the composition inhibits the proliferation of bladder epithelial cells;
(c) the antiproliferative is characterized by one or more characteristics selected from the group consisting of:
(i) a blocked N-terminal amino acid sequence;
(ii) a gly-gly-ala modified lysine segment;
(iii) the following amino acid composition: 2 asparagines/aspartic acids, 1 threonine, 2 serines, 4 glutamines/glutamic acid, 1 proline, 4 glycines, 2 alanines, 1 valine, 1 isoleucine, 2 leucines, 1 tyrosine, 1 phenylalanine, 1 lysine, 1 arginine, and at least one cysteine;
(iv) a cysteine coupled to a leucine; and
(v) a tyrosine coupled to a serine;

2. The composition of claim 1 comprising the purified antiproliferative factor.

3. The composition of claim 1 consisting essentially of the antiproliferative factor.

4. The composition of claim 1 comprising a fraction of a source of the antiproliferative factor.

5. The composition of claim 1 consisting essentially of a fraction of a source of the antiproliferative factor.

6. The composition of claim 1 comprising a functional equivalent of the antiproliferative factor.

7. The composition of claim 1 wherein the antiproliferative factor is characterized by a blocked N-terminal amino acid sequence.

8. The composition of claim 1 wherein the antiproliferative factor is characterized by a gly-gly-ala-modified lysine segment.

9. The composition of claim 1 wherein the antiproliferative factor is characterized by the following amino acid composition: 2 asparagines/aspartic acids, 1 threonine, 2 serines, 4 glutamines/glutamic acid, 1 proline, 4 glycines, 2 alanines, 1 valine, 1 isoleucine, 2 leucines, 1 tyrosine, 1 phenylalanine, 1 lysine, 1 arginine, and at least one cysteine.

10. The composition of claim 1 wherein the antiproliferative factor is characterized by a cysteine coupled to a leucine.

11. The composition of claim 1 wherein the antiproliferative factor is characterized by a tyrosine coupled to a serine.

12. The composition of claim 1 wherein the composition derives from a source comprising materials secreted from bladder epithelial cells from a subject having interstitial cystitis.

13. The composition of claim 1 wherein the composition derives from urine of a subject with interstitial cystitis.

14. The composition of claim 1 wherein the composition originates from a culture of epithelial cells originating from a bladder biopsy of one or more subjects with interstitial cystitis.

15. The antiproliferative factor of claim 14 wherein the cells are immortalized.

16. The composition of claim 1 wherein the composition exhibits antiproliferative activity measured by inhibition of ³H-thymidine or BrdU incorporation in a cell culture, as compared to a control composition.

17. The composition of claim 1 wherein the composition exhibits antiproliferative activity measured by inhibition of T24 bladder carcinoma cell proliferation, as compared to a control composition.

18. The composition of claim 1 wherein the composition exhibits absorbance at approximately 215 and 280 nm.

19. The composition of claim 1 wherein the composition is characterized by a molecular mass of about 1.7 kDa using HPLC/MS mass spectroscopy and about 2.5 kDa using MALDI-TOF mass spectroscopy.

20. The composition of claim 1 wherein the composition is characterized by stability in a freeze-thaw cycle, with less than about 26.7 % loss of activity.

21. The composition of claim 1 comprising a fragment of the antiproliferative factor.

22. The composition of claim 1 formulated as a pharmaceutical composition.

23. The composition of claim 1 obtained from a subject fulfilling the 1989 National Institute of Diabetes and Digestive and Kidney Diseases diagnostic criteria for interstitial cystitis.

24. The antiproliferative factor of claim 1 isolated by a method comprising:
(a) loading a <10,000 Dalton fraction of urine from a subject with interstitial cystitis patient onto a sepharose preparative column;
(b) eluting components of the fraction;
(c) testing each component for the ability to inhibit proliferation of bladder cells.

25. The antiproliferative factor of claim 24 wherein the ability to inhibit proliferation of bladder cells is determined by ³H-thymidine incorporation.

26. The antiproliferative factor of claim 24 wherein the bladder cells are human bladder cells.

27. The antiproliferative factor of claim 1 wherein the antiproliferative factor is isolated by a method comprising, in order, the following steps:
(a) obtaining, by ion-exchange chromatography, an active fraction of urine from a subject with interstitial cystitis;
(b) obtaining, by hydrophobic interaction chromatography, an active subfraction of the fraction of (a);
(c) isolating, by HPLC, the antiproliferative factor from the subfraction of (b).

28. The antiproliferative factor of claim 27 wherein the antiproliferative factor, when analyzed by MALDI-TOF mass spectrometry, produces a profile corresponding to the profile set forth in FIG. 8.

29. The antiproliferative factor of claim 27 wherein the antiproliferative factor, when analyzed by HPLC/MS mass spectrometry, produces a profile corresponding to the profile set forth in FIG. 7B.

30. The antiproliferative factor of claim 1 wherein the antiproliferative factor is isolated by a method comprising, in order, the following steps:
(a) obtaining a <10,000 dalton fraction of urine from a subject with interstitial cystitis;
(b) obtaining, by ion-exchange chromatography, an active sub-fraction from the fraction of (a);
(c) obtaining, by hydrophobic interaction chromatography, an active subfraction of the fraction of (b);
(d) isolating, by HPLC, the antiproliferative factor from the subfraction of (c).

31. A purified antiproliferative factor isolated from a composition comprising materials produced by bladder epithelial cells from a subject exhibiting decreased levels of heparin-binding epidermal growth factor-like growth factor, as compared to levels of heparin-binding epidermal growth factor-like growth factor in a sample of asymptomatic subjects or subjects with bacterial cystitis.

32. A purified antiproliferative factor isolated from a composition comprising materials found in urine from a subject exhibiting decreased levels of heparin-binding epidermal growth factor-like growth factor, as compared to levels of heparin-binding epidermal growth factor-like growth factor in a sample of asymptomatic subjects or subjects with bacterial cystitis.

33. A purified antiproliferative factor isolated from a composition comprising materials produced by bladder epithelial cells from a subject exhibiting increased levels of one or more factors selected from the group consisting of epidermal growth factor, insulin-like growth factor 1, and insulin-like growth factor binding protein 3, as compared to asymptomatic subjects or subjects with bacterial cystitis.

34. A purified antiproliferative factor isolated from a composition comprising materials found in urine from a subject exhibiting increased levels of one or more factors selected from the group consisting of epidermal growth factor, insulin-like growth factor 1, and insulin-like growth factor binding protein 3, as compared to asymptomatic subjects or subjects with bacterial cystitis.

35. An active fraction of urine from a subject with interstitial cystitis exhibiting antiproliferative activity.

36. The active fraction of claim 35 obtained by a method comprising a procedure selected from the group consisting of: ion-exchange chromatography, hydrophobic interaction chromatography, and HPLC.

37. A method for inhibiting epithelial cell HB-EGF production, the method comprising contacting epithelial cells with a composition of any of claims 1-36.

38. A method for identifying the structure of the antiproliferative factor of claim 1, the method comprising analyzing the structure using one-dimensional and/or two-dimensional NMR.

39. A method for downregulating HB-EGF production by a cell comprising bringing the cell into contact with a composition of any of claims 1-36.

40. A method for regulating production of HB-EGF, epidermal growth factor, insulin-like growth factor 1, and insulin-like growth factor binding protein 3 in a subject comprising administering to the subject the composition of any of claims 1-36.

41. A method for treating cancer in a subject, the method comprising administering to the subject a therapeutically effective amount of the composition of any of claims 1-36.

42. The method of claim 41 wherein the cancer is a bladder cancer.

43. The method of claim 41 wherein the cancer is a transitional cell carcinoma.

44. A method for achieving a biological effect comprising bringing a cell into contact with antiproliferative factor, wherein the effect is selected from the group consisting of:
(a) downregulation of HB-EGF production;
(b) stimulation of the production of epidermal growth factor;
(c) stimulation of the production of insulin-like growth factor 1; and
(d) stimulation of the production of insulin-like growth factor binding protein 3.

45. A method of diagnosing interstitial cystitis in a subject comprising determining if antiproliferative factor is present in the urine of said subject.

46. The method of claim 45 wherein the step for determining if antiproliferative factor is present further comprises;
(a) obtaining a <10,000 dalton fraction of urine from a subject with interstitial cystitis
(b) obtaining, by ion-exchange chromatography, an sub-fraction from the fraction of (a);
(c) obtaining, by hydrophobic interaction chromatography, an subfraction of the fraction of (b);
(d) isolating, by HPLC, antiproliferative factor from the subfraction of (c).

47. The method of claim 45 wherein the step for determining if antiproliferative factor is present further comprises
(a) incubating the urine or a portion of the urine with cells;
(b) measuring the proliferation of the cells; and
(c) comparing the amount of proliferation of the cells of (b) against the known rate of proliferation of cells not in contact with antiproliferative factor.

48. The method of claims 47 wherein the cells are selected from the group comprising normal epithelial cells, immortalized epithelial cells, cancer epithelial cells, normal bladder epithelial cells, bladder cancer epithelial cells, and immortalized bladder epithelial cells.

49. The method of claim 45 wherein the step for determining if antiproliferative factor is present further comprises
(a) incubating the urine or a portion of the urine with cells;
(b) measuring amount of BrdU incorporation into the cells; and
(c) comparing the amount of BrdU incorporation into the cells of (b) against the known rate of BrdU incorporation into cells not in contact with antiproliferative factor.

50. The method of claims 49 wherein the cells are selected from the group comprising normal epithelial cells, immortalized epithelial cells, cancer epithelial cells, normal bladder epithelial cells, bladder cancer epithelial cells, and immortalized bladder epithelial cells.

51. A method of diagnosing interstitial cystitis in a subject comprising:
(a) measuring the amount of antiproliferative factor in a urine sample from said subject; and
(b) comparing said amount with the level of antiproliferative factor in normal subjects, wherein an increase in the amount of antiproliferative factor as compared to normal subjects is indicative of interstitial cystitis.

52. The method of claim 51 wherein said measuring involves a biologic assay.

53. The method of claim 51 wherein said measuring involves an antibody-based assay.

54. The method of claim 53 wherein said antibody-based assay is selected from the group comprising an enzyme linked immunosorbent assay, a Western blot, and a radioimmunoassay.

55. A diagnostic kit for use in diagnosing interstitial cystitis comprising:
(a) a measurer of levels of antiproliferative factor in a sample of urine; and
(b) an indicator for determining if the measurement of step (a) falls in a range associated with interstitial cystitis.

56. The kit of claim 55 wherein said measurer is selected from the group comprising a biologic assay, an antibody-based assay, an enzyme linked immunosorbent assay, a Western blot, and a radioimmunoassay.

57. A diagnostic kit for use in diagnosing interstitial cystitis comprising;
(a) an aliquot of antibodies that bind to antiproliferative factor;
(b) immunoassay reagents; and
(c) a control for determining if a measurement of antiproliferative factor indicates a diagnosis of interstitial cystitis.

58. The kit of claim 57 wherein said control comprises instructions indicating that an increase in the amount of antiproliferative factor indicates a diagnosis for interstitial cystitis.
